(19) United States
(12) Patent Application Publication
(10) Pub. No.: US 2024/0238291 A1
(43) Pub. Date: Jul. 18, 2024

(54) COMBINATIONS FOR TREATMENT OF CANCER

(71) Applicants: SYNDAX PHARMACEUTICALS, INC., Waltham, MA (US); BOARD OF REGENTS, THE UNIVERSITY OF TEXAS SYSTEM, Austin, TX (US)

(72) Inventors: Gerald M. MCGEEHAN, Newtown Square, PA (US); Peter ORDENTLICH, Lexington, MA (US); Bing CARTER, Houston, TX (US); Michael ANDREEFF, Houston, TX (US)

(21) Appl. No.: 18/560,282

(22) PCT Filed: May 12, 2022
(86) PCT No.: PCT/US22/29002

§ 371 Date: Nov. 10, 2023

Related U.S. Application Data
(60) Provisional application No. 63/187,753, filed on May 12, 2021.

(51) Int. Cl. A61K 31/513; A61K 31/497; A61K 45/06; A61P 35/02

(52) U.S. Cl. CPC A61K 31/513; A61K 31/497; A61K 45/06; A61P 35/02

(57) ABSTRACT

The present disclosure is directed to combinations of menin inhibitors and Bcl-2 inhibitors, optionally in further combination with hypomethylating agents and/or FLT3 inhibitors for the treatment of cancer. Specifically, menin inhibitors combined with venetoclax are synergistic in the treatment of cancers with a HOX gene signature such as acute myeloid leukemia.

### CROSS-REFERENCE TO RELATED APPLICATIONS

This application claims priority to U.S. Provisional Application 63/187,753 filed on May 12, 2021, which is incorporated herein by reference in its entirety.

### FIELD OF THE INVENTION

The present disclosure is directed to methods of treating cancer with combinations including menin inhibitors and Bcl-2 inhibitors.

### BACKGROUND

Nucleophosmin (NPM1), encoding a primarily nucleolar localized multifunctional protein, is the most commonly mutated gene in adult acute myeloid leukemia (AML) (approximately 30%). Mutations in NPM1 result in its aberrant cytoplasmic localization (NPM1c). The interaction of mixed-lineage leukemia (MLL1) with menin in NPM1 mutated AML shares a common HOX gene signature and dependencies as that of MLL-rearrangements (MLL1-r) with menin. The inhibition of menin has demonstrated anti-leukemia activity in both NPM1c and MLL-r AML. NPM1 mutations in AML frequently occur in patients with other mutations, such as FLT3-ITD and FLT3 tyrosine kinase domain (TKD) mutations. Co-inhibition of menin and FLT3 has demonstrated enhanced anti-leukemia activity in MLL-r/FLT3- and NPM1c/FLT3-mutated AML.

Targeting B-cell lymphoma 2 (Bcl-2), a critical factor for AML cell and AML stem/progenitor cell survival, has emerged as a promising therapeutic option for patients with AML. However, despite the major improvement of combining the Bcl-2 inhibitor venetoclax with hypomethylating agents, most patients develop resistance and ultimately relapse. The present disclosure addresses these unmet clinical needs.

### SUMMARY

In some aspects, the present disclosure is directed to a method of treating cancer with a HOX gene signature in a subject in need thereof comprises administering to the subject a synergistic combination of a menin inhibitor and a Bcl-2 inhibitor. In some aspects, the present disclosure is directed to a method of treating cancer with a HOX gene signature in a subject in need thereof comprises administering to the subject a synergistic combination of a therapeutically effective amount of a menin inhibitor and a therapeutically effective amount of a Bcl-2 inhibitor. The method optionally further comprises administering a CYP3A inhibitor, an FLT3 inhibitor, a hypomethylating agent, or a combination thereof.

In some aspects, the present disclosure is directed to a therapeutic combination comprising a menin inhibitor and a Bcl-2 inhibitor. The combination optionally comprises a CYP3A inhibitor, an FLT3 inhibitor, a hypomethylating agent, or a combination thereof.

In some aspects, the present disclosure is directed to a therapeutic combination comprises a therapeutically effective amount of a menin inhibitor and a therapeutically effective amount of a Bcl-2 inhibitor. The combination optionally comprises a CYP3A inhibitor, an FLT3 inhibitor, a hypomethylating agent, or a combination thereof.

### BRIEF DESCRIPTION OF THE FIGURES

FIGS. 1A-1H: FIG. 1A is a mouse model and experimental scheme of treatment; FIGS. 1B-E: % huCD45⁺ in peripheral blood at 2 weeks (FIG. 1B) and 4 weeks (FIG. 1C) and at the end of the treatment in bone morrow (BM) (FIG. 1D) and spleen (FIG. 1E), determined by flow cytometry; FIG. 1F: Spleen weight and size at the end of the treatment; FIG. 1G: Survival curve; FIG. 1H: H&E staining of BM and spleen in each treatment groups at the end of the treatment (magnification 40×). SNDX (SNDX-50469), a menin inhibitor is Compound (I); VEN is venetoclax.

FIGS. 2A-2F: FIG. 2A: HuCD45+ cells in various treatment groups; 2B: Clusters of leukemia cells and leukemia stem/progenitor cells; FIG. 2C: % viable leukemia cells and leukemia stem/progenitor cells in each treatment groups; FIG. 2D: Protein expression in huCD45⁺ cells in various treatment groups; FIG. 2E: % HuCD11b⁺CD45⁺ cells in each treatment groups; FIG. 2F: Protein levels in CD34⁺CD38⁺ and CD34⁺CD38⁻ leukemia stem/progenitor cells in each treatment groups. Cells were collected at the end of treatments from mouse BM and protein levels were determined by CyTOF analysis. SNDX is Compound (I); VEN is venetoclax.

FIG. 3 shows the Compound (I) levels in mouse plasma after 2-wk treatment. SNDX is Compound (I); VEN is venetoclax.

FIG. 4 shows the mouse weight. SNDX is Compound (I); VEN is venetoclax.

FIG. 5 depicts metal-tagged antibodies used for cytometry by time-of flight (CyTOF) analysis.

FIG. 6A-G show the combined inhibition of menin, BCL-2, and FLT3 has strong antileukemia activity and prolongs survival in an NPM1c/FLT3-ITD/TKD PDX model. (6A) The experimental scheme. (B-E) Percentages of HuCD45⁺ cells in the peripheral blood at 2 weeks (6B) and 4 weeks (6C) and in the spleen (6D) and BM (6E) at the end of treatment, as determined by flow cytometry. Spleens harvested at the end of the treatment are also shown in (6D). (6F) Survival by treatment type. Mouse survival was estimated using the Kaplan-Meier method, and survival data were analyzed using the log-rank test. (6G) Immunohistochemical staining for HuCD45. Left, Immunohistochemical staining for HuCD45 in BM cells from a PDX-bearing NSG mouse (positive control) and BM cells from a non-PDX-bearing NSG mouse (negative control). Right, Immunohistochemical staining for HuCD45 in lung, liver, and heart tissues from a mouse treated with the 4-drug combination (marked * in [F]). Differences between groups were determined using the Student t-text. P values≤0.05 were considered statistically significant. * P≤0.05; ** P≤0.01; *** P≤0.001; **** P≤0.0001. d, day; wk, week; PB, peripheral blood; SNDX, SNDX-50469; Gil, gilteritinib; VEN, venetoclax; 5-AZA, 5-azacitidine.

FIGS. 7A-D show menin, FLT3, and/or BCL-2 inhibition targets leukemia cells and stem/progenitor cells and modulates HOX targets and BCL-2 protein levels in BM. PhenoGraph was used to cluster cell populations according to cell surface marker expression. Cisplatin-low viable single cells were gated with FlowJo software (version 10.7, FlowJo LLC) and exported as flow cytometry standard (FCS) data for subsequent analysis in Cytofkit. Cell populations identified and embedded by PhenoGraph in the “Cytofkit_analyzedFCS” files were gated in FlowJo to quantify marker expression. ArcSinh-transformed counts for each protein expression in desired cell populations were visualized with heat maps. (7A) Clusters of leukemia cells and leukemia stem/progenitor cells. (7B) Percentages of viable leukemia cells and leukemia stem/progenitor cells in each treatment group. (7C) HuCD45 cells in the treatment groups. (7D) Protein expression in huCD45⁺ cells in the treatment groups. CON, control, SNDX, SNDX-50469; Gil, gilteritinib; VEN, venetoclax.

### DETAILED DESCRIPTION

Provided herein are therapeutic combinations and compositions comprising a menin inhibitor and a Bcl-2 inhibitor, optionally further comprising an FLT3 inhibitor, a hypomethylating agent, or a combination thereof. Further provided are methods for administering such combinations and compositions for the treatment of cancer, specifically cancers with a HOX gene signature.

In an aspect, the therapeutic combinations and compositions comprising a menin inhibitor and a Bcl-2 inhibitor further including a hypomethylating agent. As shown herein, the addition of the hypomethylating agent 5-azacitidine to the menin inhibitor and Bcl-2 inhibitor extended survival, and this combination potentially eliminated leukemia in an art-accepted mouse model.

In a further aspect, the further addition of an FLT3 inhibitor such as gilteritinib to the combinations and compositions further extended survival in an art-accepted mouse model of AML.

Unless defined otherwise, all technical and scientific terms used herein have the same meaning as is commonly understood by one of skill in the art to which the claimed subject matter belongs. It is to be understood that the following detailed description is exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. In this application, the use of “or” means “and/or” unless stated otherwise.

Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### Therapeutic Combinations

In one aspect, provided herein are therapeutic combinations comprising a menin inhibitor and a Bcl-2 inhibitor, optionally further comprising an FLT3 inhibitor, a hypomethylating agent, or a combination thereof. The menin inhibitor, the Bcl-2 inhibitor, the FLT3 inhibitor and the hypomethylating agent may be present in one or more pharmaceutical compositions.

Menin inhibitors include 5-fluoro-N,N-diisopropyl-2-((4-(7-((trans-4-(methylsulfonamido)cyclohexyl)methyl)-2,7-diazaspiro[3.5]nonan-2-yl)pyrimidin-5-yl)oxy)benzamide, N-ethyl-2-((4-(7-((trans-4-(ethylsulfonamido)cyclohexyl)methyl)-2,7-diazaspiro[3.5]nonan-2-yl)pyrimidin-5-yl)oxy)-5-fluoro-N-isopropylbenzamide, JNJ-75276617, KO-539, DS-1594b, DSP-5336, a pharmaceutically acceptable salt thereof, or a combination thereof.

An exemplary menin inhibitor is 5-fluoro-N,N-diisopropyl-2-((4-(7-(((1r,4r)-4-(methylsulfonamido)cyclohexyl)methyl)-2,7-diazaspiro[3.5]nonan-2-yl)pyrimidin-5-yl)oxy)benzamide (Compound I; SNDX-50469), or a pharmaceutically acceptable salt, stereoisomer, geometric isomer or tautomer thereof. Another exemplary menin inhibitor is N-ethyl-2-((4-(7-(((1r,4r)-4-(ethylsulfonamido)cyclohexyl) methyl)-2,7-diazaspiro[3.5]nonan-2-yl)pyrimidin-5-yl)oxy)-5-fluoro-N-isopropylbenzamide (Compound II; SNDX-5613), or a pharmaceutically acceptable salt, stereoisomer, geometric isomer or tautomer thereof. In some embodiments, the menin inhibitor of Compound (I) or Compound (II) embodies stereoisomers, geometric isomers and/or tautomers. In some embodiments, the menin inhibitor used in a therapeutic combination provided herein is selected from Compound (I) and Compound (II):

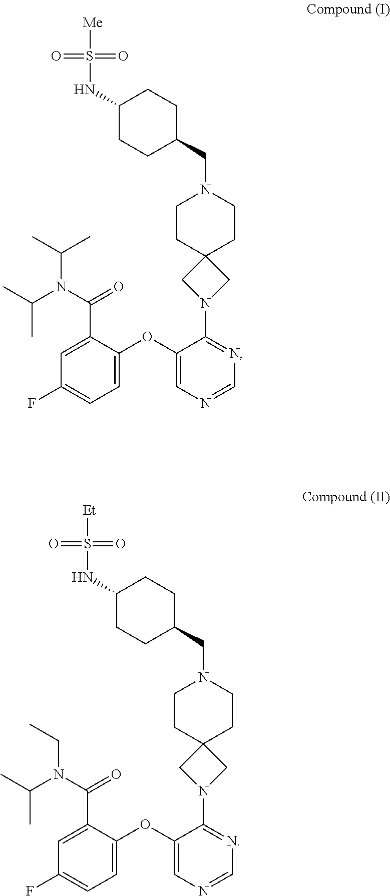 [SMILES: CC(C)N(C(=O)c1cc(F)ccc1Oc1cncnc1N1CC2(CCN(C[C@H]3CC[C@H](NS(C)(=O)=O)CC3)CC2)C1)C(C)C ; CCN(C(=O)c1cc(F)ccc1Oc1cncnc1N1CC2(CCN(C[C@H]3CC[C@H](NS(=O)(=O)CC)CC3)CC2)C1)C(C)C]

or a pharmaceutically acceptable salt, stereoisomer, geometric isomer or tautomer thereof.

In some embodiments, the pharmaceutically acceptable salt of Compound (I) or Compound (II) is a bis-methanesulfonic acid salt. In some embodiments, the pharmaceutically acceptable salt is a bis-hydrochloric acid salt. In some embodiments, the pharmaceutically acceptable salt is a sesquifumaric acid salt.

In some embodiments, the menin inhibitor of Compound (I) or Compound (II) may be administered at a dose of 276 mg/day without a strong CYP3A4 inhibitor and 163 mg/day with strong CYP3A4 inhibitor. The menin inhibitor of Compound (I) or Compound (II) may be administered once or twice per day.

Additional menin inhibitors known in the art include JNJ-75276617, KO-539, BMF-219, DSP-5336, ISC-30, the antibody A300-105A (commercially available from Bethyl Laboratories), MI-0202, MI-503, MI-463, MI-136, ML-227, and DS-1594. Menin inhibitors are described in U.S. Pat. Nos. 11,220,517; 10,174,041; 10,752,639; and 11,236,106, U.S. Patent Application Publication Nos. US 2021/0115018, US 2019/0307750, US 2016/0339035, and PCT Application Publication Nos. WO 2017/112768, WO 2017/214367, WO 2018/053267, WO 2020/069027, WO 2021/207335, incorporated herein by reference for their disclosure of menin inhibitors.

A wide variety of pharmaceutically acceptable salts may be formed from the menin inhibitor and include: acid addition salts formed by reacting the menin inhibitor with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, benzoic acid, cinnamic acid, mandelic acid, methanesulfonic acid, ethanesulfonic acid, p-toluenesulfonic acid, salicylic acid, and the like; acid addition salts formed by reacting the menin inhibitor with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to the menin inhibitor refers to a salt of the menin inhibitor, which does not cause significant irritation to a mammal to which it is administered and does not substantially abrogate the biological activity and properties of the compound.

Also included herein are solvates of Compound (I) and Compound (II). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. Hydrates are formed when the solvent is water, or alcoholates are formed when the solvent is alcohol.

In yet other embodiments, the menin inhibitor, or a pharmaceutically acceptable salt thereof, is prepared in various forms, including but not limited to, amorphous phase, crystalline forms, milled forms and nano-particulate forms. In some embodiments, the menin inhibitor, or a pharmaceutically acceptable salt thereof, is amorphous. In some embodiments, the menin inhibitor, or a pharmaceutically acceptable salt thereof, is amorphous and anhydrous. In some embodiments, the menin inhibitor, or a pharmaceutically acceptable salt thereof, is crystalline. In some embodiments, the menin inhibitor, or a pharmaceutically acceptable salt thereof, is crystalline and anhydrous.

The synergistic combinations described herein include a menin inhibitor and a Bcl-2 inhibitor. Exemplary Bcl-2 inhibitors include venetoclax, navitoclax, obatoclax, subatoclax, maritoclax, S64315, oblimersen, or other agents targeting antiapoptotic Bcl-2 family proteins, and combinations thereof. In some embodiments, the Bcl-2 inhibitor is venetoclax.

In some embodiments, the combination of the menin inhibitor and the Bcl-2 inhibitor acts synergistically against cancer, specifically cancer with a HOX gene signature. For example, the combination of the menin inhibitor and the Bcl-2 inhibitor may decrease the number of leukemia cells in the blood, spleen and/or bone marrow of a subject to a greater degree than either the menin inhibitor or the Bcl-2 inhibitor alone. In some embodiments, the menin inhibitor or the Bcl-2 inhibitor alone do not substantially decrease the amount of leukemia cells in the blood, spleen and/or bone marrow of a subject, but the combination of the menin inhibitor and the Bcl-2 inhibitor does substantially decrease the number of leukemia cells in the blood, spleen and/or bone marrow of the subject. “Substantial” in the context of a change (e.g., an increase or decrease) of a clinical endpoint (e.g., number of leukemia cells in the blood, or expression of a protein) means a clinically relevant or statistically significant change (e.g., a change of at least 5%). The number of leukemia cells in a tissue (e.g., the blood, spleen or bone marrow) may be determined, for example, by measuring the number of human CD45⁺ cells in said tissue using flow cytometry. In some embodiments, the subject is a human subject treated in accordance with a method described herein. In some embodiments, the subject has one or more AML mutations, (e.g., a NPM1c, FLT3-ITD, and/or FLT3-TKD).

The combination of the menin inhibitor and the Bcl-2 inhibitor may also synergistically prolong the survival of a subject with cancer, specifically a cancer with a HOX gene signature (e.g., the cancer has one or more AML mutations, a NPM1c, FLT3-ITD, and/or FLT3-TKD). For example, the combination of the menin inhibitor and the Bcl-2 inhibitor may prolong the survival of a cancer patient (e.g., a NPM1c, FLT3-ITD, and/or FLT3-TKD) to a greater degree than either the menin inhibitor or the Bel-2 inhibitor alone. In some embodiments, the menin inhibitor or the Bcl-2 inhibitor alone do not substantially prolong survival of a subject with one or more AML mutations (e.g., a NPM1c, FLT3-ITD, and/or FLT3-TKD), but the combination of the menin inhibitor and the Bcl-2 inhibitor does substantially prolong the survival of the subject with one or more AML mutations (e.g., a NPM1c, FLT3-ITD, and/or FLT3-TKD).

In some embodiments, the combination of the menin inhibitor and the Bcl-2 inhibitor synergistically increases the expression of pro-apoptotic proteins (e.g., Bim) in a subject (e.g., in CD34⁺CD38⁺ cells of a subject). In some embodiments, the combination of the menin inhibitor and the Bcl-2 inhibitor synergistically decreases the expression of anti-apoptotic proteins (e.g., Bc1-2 and/or Bcl-xL) in a subject (e.g., in CD34⁺CD38⁺ cells of a subject). In some embodiments, the combination of the menin inhibitor and the Bcl-2 inhibitor synergistically decrease the expression of proteins associated with resistance to treatment with Bcl-2 inhibitors (e.g., Bcl-2A1) in a subject (e.g., in human CD45⁺ cells). The expression of proteins may be determined using any suitable method known in the art or described herein including, for example, flow cytometry, immunohistochemistry, or Western Blotting. Suitable samples in which protein expression can be analyzed include, without limitation, the blood, bone marrow and the spleen. In some embodiments, the subject is a human subject treated in accordance with the methods described herein. In some embodiments, the subject has a cancer with one or more AML mutations (e.g., a NPM1c, with or without FLT3-ITD, and/or TKD). In some embodiments, the synergistic increase in pro-apoptotic proteins, the synergistic decrease in anti-apoptotic proteins, and/or the synergistic decrease in proteins associated with resistance to treatment with Bcl-2 inhibitors is measured in the CD34⁺CD38⁺ subject. In some embodiments, the synergistic increase in pro-apoptotic proteins, the synergistic decrease in anti-apoptotic proteins, and/or the synergistic decrease in proteins associated with resistance to treatment with Bcl-2 inhibitors is more pronounced in CD34⁺CD38⁺ cells compared to CD34⁺CD38⁻ cells in a subject.

In some embodiments, the combination of the menin inhibitor and the Bcl-2 inhibitor enhances, increases or prolongs either potency or duration of therapeutic effect of the menin inhibitor.

In an aspect, the combination of the menin inhibitor and the Bel-2 inhibitor further comprises a hypomethylating agent. Exemplary hypomethylating agents include azacitidine, decitabine, guadecitabine, and combinations thereof. The hypomethylating agent can be administered simultaneously or sequentially with the menin inhibitor and the Bcl-2 inhibitor.

In an aspect, the combination of the menin inhibitor and the BCL-2 inhibitor further comprises an FLT3 inhibitor. In another aspect, the combination of the menin inhibitor, the Bcl-2 inhibitor, and the hypomethylating agent further comprises an FLT3 inhibitor. Exemplary FLT3 inhibitors include midostaurin, sorafenib, sunitinib, lestaurtinib, tandutinib, gilteritinib, quizartinib, crenolanib, and combinations thereof.

The FLT3 inhibitor can be administered simultaneously or sequentially with the menin inhibitor and the BCL-2 inhibitor.

In some embodiments, a subject treated with a therapeutic combination provided herein is further administered a cytochrome P450 3A (CYP3A) inhibitor, e.g., a CYP3A4 inhibitor. Cytochrome P450 enzymes modify a variety of substrates. The modifications include hydroxylation, epoxidation, aromatic oxidations, heteroatom oxidations, N- and O-dealkylations, aldehyde oxidations, and dehydrogenations. In some embodiments, the combination of the menin inhibitor, the Bcl-2 inhibitor, and the CYP3A4 inhibitor acts synergistically to treat cancer.

Without wishing to be bound by theory, the administration of the CYP3A inhibitor (e.g., a CYP3A4 inhibitor) is believed to slow the metabolism of the menin inhibitor and/or the Bcl-2 inhibitor. Thus, in some embodiments, the administration of the CYP3A inhibitor (e.g., a CYP3A4 inhibitor) increases plasma levels of the menin inhibitor and/or the Bcl-2 inhibitor. In some embodiments, the administration of the CYP3A inhibitor (e.g., a CYP3A4 inhibitor) increases the oral bioavailability of the menin inhibitor and/or the Bcl-2 inhibitor. In some embodiments, the administration of the CYP3A inhibitor (e.g., a CYP3A4 inhibitor) increases the Cₘₐₓ of the menin inhibitor and/or the Bcl-2 inhibitor. In some embodiments, the administration of the CYP3A inhibitor (e.g., a CYP3A4 inhibitor) increases the AUC of the menin inhibitor and/or the Bcl-2 inhibitor. In some embodiments, the administration of the CYP3A inhibitor (e.g., a CYP3A4 inhibitor) increases the Tin of the menin inhibitor and/or the Bc1-2 inhibitor.

In some embodiments, the administration of the CYP3A inhibitor (e.g., a CYP3A4 inhibitor) enhances the efficacy of the menin inhibitor and/or the Bcl-2 inhibitor to treat a variety of diseases. In some embodiments, administration of the CYP3A inhibitor (e.g., a CYP3A4 inhibitor) enhances, increases, and/or prolongs the efficacy or duration of the menin inhibitor's therapeutic effect and/or of the Bcl-2 inhibitor's therapeutic effect.

In some embodiments, the CYP3A inhibitor is a CYP3A4 inhibitor. In some embodiments, the CYP3A inhibitor is a CYP3A5 inhibitor. In some embodiments, the CYP3A inhibitor is a CYP3A7 inhibitor.

In some embodiments, the therapeutic combination comprising the menin inhibitor and the Bcl-2 inhibitor is therapeutically effective at a lower dose when combined with the CYP3A inhibitor (e.g., a CYP3A4 inhibitor). In some embodiments, the therapeutic combination comprising the menin inhibitor and the Bcl-2 inhibitor is more effective in combination with a CYP3A inhibitor (e.g., a CYP3A4 inhibitor).

In some embodiments, the CYP3A4 inhibitor is: an anti-arrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A4 inhibitor is: alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; troleandromycin, verapamil; telaprevir; vincristine; voriconazole; or any combinations thereof. In some embodiments, the CYP3A4 inhibitor is cobicistat (GS-9350) or analogs or derivatives of cobicistat (GS-9350). In some embodiments, the CYP3A4 inhibitor is ketoconazole. In some embodiments, the CYP3A4 inhibitor is ritonavir.

In some embodiments, the menin inhibitor is Compound (I) and the CYP3A4 inhibitor is an azole antifungal. In some embodiments, the menin inhibitor is compound (II) and the CYP3A4 inhibitor is an azole antifungal.

In some embodiments, the menin inhibitor is Compound (I) and the CYP3A4 inhibitor is posaconazole. In some embodiments, the menin inhibitor is Compound (II) and the CYP3A4 inhibitor is posaconazole.

In some embodiments, the menin inhibitor is administered in combination with a CYP3A4 inducer. In some embodiments, CYP3A4 inducers include but are not limited to one or more of avasimibe, phenytoin, carbamazepine, rifampin, enzalutamide, and St John's wort.

### Doses and Administration

The menin inhibitor and the Bcl-2 inhibitor of the therapeutic combination provided herein may be administered in the same composition or in separate compositions.

The menin inhibitor and the Bcl-2 inhibitor may be administered simultaneously or sequentially. In some embodiments, the menin inhibitor and the Bcl-2 inhibitor are administered in temporal proximity.

The menin inhibitor and the Bcl-2 inhibitor may be administered at the same frequency or at different frequencies. In some embodiments, the first administration of the menin inhibitor and the first administration of the Bcl-2 inhibitor occurs in temporal proximity.

In some embodiments, “temporal proximity” means that administration of one therapeutic agent occurs within a time period before or after the administration of another therapeutic agent, such that there is a synergistic effect between the one therapeutic agent and the other therapeutic agent (e.g., between the menin inhibitor and the Bcl-2 inhibitor). “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some embodiment, the menin inhibitor is administered daily, every 2 days, every 3 days, every 4 days, every 5 days, every 6 days, or weekly. In some embodiments, the Bcl-2 inhibitor is administered daily, every 2 days, every 3 days, every 4 days, every 5 days, every 6 days, or weekly. In some embodiments, the menin inhibitor is administered more than once a day, e.g., every 4 hours, every 6 hours, or every 12 hours.

In some embodiments, the menin inhibitor and the Bel-2 inhibitor are administered concurrently. In some embodiments, the menin inhibitor and the Bcl-2 inhibitor are administered simultaneously, essentially simultaneously or within the same treatment protocol.

In some embodiments, the daily dosage of the menin inhibitor is between about 150 mg and about 200 mg, between about 200 mg and about 250 mg; between about 250 mg and about 300 mg; between about 300 mg and about 350 mg; between about 350 mg and about 400 mg; between about 400 mg and about 450 mg; between about 450 mg and about 500 mg; between about 500 mg and about 550 mg; between about 550 mg and about 600 mg; I between about 600 mg and about 650 mg; or between about 650 mg and about 700 mg.

In some embodiments, the daily dosage amount of the menin inhibitor is about 226 mg, 452 mg, 113 mg 326 mg or 552 mg.

In some embodiments, a dose is given once a day, given twice a day, given three times per day, given four times per day to equal the daily dose. In some embodiments, the menin inhibitor is given every 12 hours. In some embodiments, the menin inhibitor is administered at a unit dose of 113 mg. In some embodiments, the unit dose is given once a day, given twice a day, given three times per day, given four times per day. In some embodiments, one-unit dose is given per day, two-unit doses are given per day, three-unit doses are given per day, four-unit doses are given per day. In some embodiments, two-unit doses are given twice per day.

In some embodiments, the amount of the menin inhibitor that is administered is about 150, 160, 170, 180, 190, 200, 210, 220, 230, 240 250, 260, 270, 280, 290, 300, 310, 320, 330, 340, 350, 360, 370, 380, 390, 400, 410, 420, 430, 440, 450, 460, 470, 480, 490, 500, 510, 520 530, 540, 550, 560, 570, 580, 590, 600, 610, 620, 630, 640, 650, 670, 680, 690 or 700 mg/day. In some embodiments, the daily dosage is divided into multiple administrations and is given once a day, given twice a day, given three times per day, given four times per day. In some embodiments, the menin inhibitor is administered once per day, twice per day, three times per day. In some embodiments, the menin inhibitor is administered once per day. In some embodiments, the menin inhibitor is administered twice per day.

In some embodiments, the menin inhibitor is administered at 50 mg QD. 113 mg QD, 113 mg q12 h, 163 mg q12 h, 226 mg q12 h, 276 mg q12 h, 339 mg q12 h, 452 mg q12 h, or 565 mg q12 h. In some embodiments, the menin inhibitor is Compound I and is administered at 50 mg QD. 113 mg QD, 113 mg q12 h, 163 q12 h, 226 mg q12 h, 276 mg q12 h, 339 mg q12 h, 452 mg q12 h, or 565 mg q12 h. In some embodiments, the menin inhibitor is a compound of Compound II and is administered at 50 mg QD, 113 mg QD, 113 mg q12 h, 163 mg q12 h, 226 mg q12 h, 276 mg q12 h, 339 mg q12 h, 452 mg q12 h, or 565 mg q12 h. In some embodiments, the menin inhibitor is a pharmaceutical formulation comprising Compound II and is administered at 50 mg QD, 113 mg QD, 113 mg q12 h, 163 mg q12 h, 226 mg q12 h, 276 mg q212 h, 339 mg q12 h, 452 mg q12 h, or 565 mg q12 h. In some embodiments, the menin inhibitor is a capsule comprising Compound II and is administered at 50 mg QD, 113 mg QD, 113 mg q12 h, 163 mg q12 h, 226 mg q12 h, 276 mg q12 h, 339 mg q12 h, 452 mg q12 h, or 565 mg q12 h. In specific embodiments, the menin inhibitor is administered every 12 hours (q12 h) at a dose of 113 mg. In specific embodiments, the menin inhibitor is administered every 12 hours (q12 h) at a dose of 163 mg. In specific embodiments, the menin inhibitor is administered every 12 hours (q12 h) at a dose of 276 mg.

In some embodiments, the daily dose of the Bcl-2 inhibitor is between about 10 mg and about 20 mg. In some embodiments, the daily dose of the Bel-2 inhibitor is between about 20 mg and about 30 mg. In some embodiments, the daily dose of the Bcl-2 inhibitor is between about 30 mg and about 40 mg. In some embodiments, the daily dose of the Bcl-2 inhibitor is between about 40 mg and about 50 mg. In some embodiments, the daily dose of the Bcl-2 inhibitor is between about 50 mg and about 60 mg. In some embodiments, the daily dose of the Bc1-2 inhibitor is between about 60 mg and about 70 mg. In some embodiments, the daily dose of the Bcl-2 inhibitor is between about 70 mg and about 80 mg. In some embodiments, the daily dose of the Bcl-2 inhibitor is between about 80 mg and about 90 mg. In some embodiments, the daily dose of the Bcl-2 inhibitor is between about 90 mg and about 100 mg. In some embodiments, the daily dose of the Bcl-2 inhibitor is between about 100 mg and about 150 mg. In some embodiments, the daily dose of the Bcl-2 inhibitor is between about 150 mg and about 200 mg. In some embodiments, the daily dose of the Bcl-2 inhibitor is between about 200 mg and about 250 mg. In some embodiments, the daily dose of the Bel-2 inhibitor is between about 250 mg and about 300 mg. In some embodiments, the daily dose of the Bcl-2 inhibitor is between about 300 mg and about 350 mg. In some embodiments, the daily dose of the Bcl-2 inhibitor is between about 350 mg and about 400 mg. In some embodiments, the daily dose of the Bcl-2 inhibitor is between about 400 mg and about 450 mg. In some embodiments, the daily dose of the Bcl-2 inhibitor is between about 450 mg and about 500 mg.

In some embodiments, the daily dose of the Bcl-2 inhibitor is about 20 mg. In some embodiments, the daily dose of the Bcl-2 inhibitor is about 50 mg. In some embodiments, the daily dose of the Bcl-2 inhibitor is about 100 mg. In some embodiments, the daily dose of the Bc1-2 inhibitor is about 200 mg. In some embodiments, the daily dose of the Bcl-2 inhibitor is about 400 mg.

In some embodiments, the daily dose of the Bcl-2 inhibitor is 20 mg for a first week, 50 mg for a second week, 100 mg for a third week, 200 mg for a fourth week and dose of 400 mg for a fifth week and subsequent weeks.

In some embodiments, a subject treated with a therapeutic combination provided herein is further administered a CYP3A inhibitor (e.g., a CYP3A4 inhibitor). Any suitable daily dose of a CYP3A4 inhibitor is contemplated for use with the compositions, dosage forms, and methods disclosed herein. For example, the daily dose of the CYP3A4 inhibitor depends of the strength of the CYP3A4 inhibitor. Weak CYP3A4 inhibitors (e.g. cimetidine) will require higher daily doses than moderate CYP3A4 inhibitors (e.g., erythromycin, grapefruit juice, verapamil, diltiazem), and moderate CYP3A4 inhibitors will require higher daily doses than strong CYP3A4 inhibitors (e.g., indinavir, nelfmavir, ritonavir, clarithromycin, itraconazole, ketoconazole, nefazodone).

The menin inhibitor, the Bcl-2 inhibitor, and the CYP3A inhibitor (e.g., CYP3A4 inhibitor) may be administered in the same composition, in separate compositions, simultaneously, sequentially, in temporal proximity, at the same frequency or at different frequencies.

In some embodiments, the daily dose of the CYP3A4 inhibitor that is administered in combination with the therapeutic combination comprising a menin inhibitor and a Bcl-2 inhibitor is from 50 mg/day up to, and including, 1000 mg/day. In some embodiments, each dose is given once a day, given twice a day, given three times per day, given four times per day. In some embodiments, the CYP3A4 dosage is dependent on the specific CYP3A4 inhibitor. In some embodiments, the daily dosage of each CYP3A4 inhibitor is administered according to approved labeling for other indications. In some embodiments, the amount of the CYP3A4 inhibitor that is administered is about 40, 50, 60, 70, 80, 90, 100, 110, 120, 130, 140 150, 160, 170, 180, 190, 200, 210, 220, 230, 240 250, 260, 270, 280, 290, 300, 310, 320, 330, 340, 350, 360, 370, 380, 390, 400, 410, 420, 430, 440, 450, 460, 470, 480, 490, 500, 510, 520 530, 540, 550, 560, 570, 580, 590, 600, 610, 620, 630, 640, 650, 670, 680, 690 or 700 mg/day. In some embodiments, the daily dose divided and given once a day, given twice a day, given three times per day, or given four times per day.

In some embodiments, a subject treated with a therapeutic combination provided herein is further administered an FLT3 inhibitor. Any suitable daily dose of an FLT3 inhibitor is contemplated for use with the compositions, dosage forms, and methods disclosed herein.

The menin inhibitor, the Bcl-2 inhibitor, the CYP3A inhibitor (e.g., CYP3A4 inhibitor), and the FLT3 inhibitor may be administered in the same composition or in separate compositions.

The menin inhibitor, the Bcl-2 inhibitor, the CYP3A inhibitor (e.g., CYP3A4 inhibitor) and the FLT3 inhibitor may be administered simultaneously or sequentially. In some embodiments, the menin inhibitor, the Bcl-2 inhibitor, the CYP3A inhibitor (e.g., CYP3A4 inhibitor), and the FLT3 inhibitor are administered in temporal proximity.

The menin inhibitor, the Bcl-2 inhibitor, the CYP3A inhibitor (e.g., CYP3A4 inhibitor), and the FLT3 inhibitor may be administered at the same frequency or at different frequencies. In some embodiments, the first administration of the menin inhibitor, the first administration of the Bc1-2 inhibitor, the first administration of the CYP3A inhibitor (e.g., CYP3A4 inhibitor), and the first administration of the FLT3 inhibitor occurs in temporal proximity.

In some embodiments, the daily dose of the FLT3 inhibitor or the hypomethylation agent that is administered in combination with the therapeutic combination comprising a menin inhibitor, a Bcl-2 inhibitor and optionally a CYP3A inhibitor (e.g., CYP3A4 inhibitor) is from 50 mg/day up to, and including. 1000 mg/day. In some embodiments, each dose is given once a day, given twice a day, given three times per day, given four times per day. In some embodiments, the FLT3 inhibitor dosage is dependent on the specific FLT3 inhibitor. In some embodiments, the hypomethylation agent dosage is dependent on the specific hypomethylation agent. In some embodiments, the daily dosage of each FLT3 inhibitor is administered according to approved labeling for other indications. In some embodiments, the amount of the FLT3 inhibitor that is administered is about 40, 50, 60, 70, 80, 90, 100, 110, 120, 130, 140, 150 160, 170, 180, 190, 200, 210, 220, 230, 240, 250, 260, 270, 280, 290, 300, 310, 320, 330, 340, 350, 360, 370, 380, 390, or 400 mg/day. In some embodiments, the FLT3 inhibitor and/or the hypomethylation agent is given once a day, given twice a day, given three times per day, given four times per day.

In some embodiments, the menin inhibitor, the Bcl-2 inhibitor, the CYP3A inhibitor (e.g., CYP3A4 inhibitor) and/or hypomethylation agent and/or the FLT3 inhibitor are co-administered (e.g., in a single dosage form or in separate dosage forms), once per day. In some embodiments, the menin inhibitor is administered twice per day and the Bcl-2 inhibitor, the CYP3A inhibitor (e.g., CYP3A4 inhibitor), and/or the hypomethylation agent and/or FLT3 inhibitor are administered (e.g., in a single dosage form or in separate dosage forms), four times per day. In some embodiments, the menin inhibitor is administered twice per day and the Bcl-2 inhibitor, the CYP3A inhibitor (e.g., CYP3A4 inhibitor) and/or the hypomethylation agent and/or FLT3 inhibitor are administered (e.g., in a single dosage form or in separate dosage forms), twice per day. In some embodiments, the menin inhibitor, the Bcl-2 inhibitor, the CYP3A inhibitor (e.g., CYP3A4 inhibitor) and/or the hypomethylation agent and/or FLT3 inhibitor are maintenance therapy. In some embodiments, the menin inhibitor is maintenance therapy.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered as a maintenance therapy, for example for a patient in remission.

In the case wherein the patient's status does not improve, the administration of the compounds may be given continuously; alternatively, the dose of drug being administered may be increased for a certain length of time. The length of the drug increase can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose increase may be from 10%-200%, including, by way of example only, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%.

If improvement of the patient's conditions has not occurred, the dosage or the frequency of administration, or both, can be increased, as a function of the symptoms, to a level at which the improved disease, disorder or condition is achieved.

In the case wherein the patient's status does improve, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens described herein can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Pharmaceutical Compositions

In one embodiment, provided herein is a pharmaceutical composition comprising a menin inhibitor, a Bcl-2 inhibitor, optionally an FLT3 inhibitor, optionally a hypomethylating agent and optionally a pharmaceutically acceptable carrier. In one embodiment, provided herein is a pharmaceutical composition comprising a menin inhibitor and a CYPR3A4 inhibitor and optionally a pharmaceutically acceptable carrier. In another embodiment, provided herein is a pharmaceutical composition comprising a menin inhibitor, a Bcl-2 inhibitor, a CYP3A inhibitor (e.g., a CYP3A4 inhibitor), optionally an FLT3 inhibitor, optionally a hypomethylating agent, and optionally a pharmaceutically acceptable carrier. The pharmaceutical compositions of the present application comprise a therapeutically effective amount of a compound (e.g., the menin inhibitor, the Bcl-2 inhibitor, and/or the CYP3A4 inhibitor, and/or the FLT3 inhibitor, and/or the hypomethylating agent) of the present application formulated together with one or more pharmaceutically acceptable carriers.

Pharmaceutical compositions including the individual compounds of the therapeutic combinations described herein in free form or in a pharmaceutically acceptable salt form in association with at least one pharmaceutically acceptable carrier or diluent may be manufactured in a conventional manner by mixing, granulating or coating methods.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers.

Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The therapeutic combinations of the application may be administered as pharmaceutical compositions by any conventional route, in particular enterally, e.g., orally, e.g., in the form of tablets or capsules, or parenterally, e.g., in the form of injectable solutions or suspensions, or topically, e.g., in the form of lotions, gels, ointments or creams, or in a nasal or suppository form.

For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The pharmaceutical compositions of this application may be administered to humans and other animals orally, rectally, parenterally, intracisternally, intravaginally, intraperitoneally, topically (as by powders, ointments, or drops), buccally, or as an oral or nasal spray.

As used herein, the term “pharmaceutically acceptable carrier” means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Further disclosed herein, in some embodiments, are dosage forms comprising the menin inhibitor, the Bcl-2 inhibitor and/or a CYP3A4 inhibitor. In some embodiments, the dosage form is a combined dosage form. In some embodiments, the dosage form is a solid oral dosage form. In some embodiments, the dosage form is a tablet, pill, or capsule. In some embodiments, the dosage form is a controlled release dosage form, delayed release dosage form, extended release dosage form, pulsatile release dosage form, multiparticulate dosage form, or mixed immediate release and controlled release formulation. In some embodiments, the dosage form comprises a controlled release coating. In some embodiments, the dosage forms comprise a first controlled release coating which controls the release of the menin inhibitor and a second controlled release coating which controls the release of the CYP3A4 inhibitor.

The active compounds may also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

### Methods of Treatment

In another aspect, provided herein are methods of treating cancer in a subject, the method comprising administering a therapeutic combination described herein.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition.

As used herein, the term “preventing,” “prevent,” or “protecting against” describes reducing or eliminating the onset of the symptoms or complications of such disease, condition or disorder.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

A method of treating cancer with a HOX gene signature in a subject in need thereof comprises administering to the subject a synergistic combination of a therapeutically effective amount of a menin inhibitor and a therapeutically effective amount of a Bcl-2 inhibitor, and optionally a therapeutically effective amount of a hypomethylating agent and/or an FLT3 inhibitor. As used herein, a HOX gene signature is set of genes that their expressions are altered—driven by altered HOX gene expression that affect initiation, development, progression of cancer, or a combination thereof. HOX gene signatures are well-known in the art. In some embodiments, the combination comprises a menin inhibitor, a Bcl-2 inhibitor, a CYP3A inhibitor (e.g., a CYP3A4 inhibitor) and optionally a therapeutically effective amount of a hypomethylating agent and/or an FLT3 inhibitor. The combination of the menin inhibitor and the Bc1-2 inhibitor and optionally the CYP3A4 inhibitor optionally further comprises a therapeutically effective amount of a hypomethylating agent, a therapeutically effective amount of an FLT3 inhibitor, or a combination thereof. The two, three, four or five agent combinations can be administered simultaneously or sequentially, and by the same or different modes of administration, e.g., oral, parenteral, and the like.

Homeobox (HOX) transcription factors are a conserved family of transcription factors. Mutations or activations in the HOX genes can lead to an increased risk of cancer, as well as affecting cancer development and/or progression. HOX gene alterations play a role in angiogenesis, autophagy, differentiation, apoptosis, proliferation, invasion, metastasis and metabolism. Cancers with a HOX gene signature include breast cancer, multiple myeloma, ovarian cancer, renal cancer, colon cancer, colorectal cancer, prostate cancer, gastric cancer, non-small cell lung cancer, glioblastoma, cervical cancer, chondrosarcoma, osteosarcoma, neuroblastoma, and hematological malignancies such as leukemias.

The term hematological malignancy includes lymphoma (e.g., non-Hodgkin's lymphomas), leukemia (e.g., AML) and multiple myeloma. Leukemias include AML, myeloid dysplastic syndromes (MDS), myeloproliferative diseases, acute lymphocytic leukemia (ALL), chronic myeloid leukemia (CML) and chronic lymphocytic leukemia (CLL). The combinations and compositions described herein are particularly useful to treat hematological malignancies.

Exemplary leukemias and lymphomas treatable by the combinations described herein include leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, ALL (also referred to as acute lymphoblastic leukemia or acute lymphoid leukemia), AML (also referred to as acute myelogenous leukemia or acute myeloblastic leukemia), acute granulocytic leukemia, acute nonlymphocytic leukemia, CLL (also referred to as chronic lymphoblastic leukemia), CML (also referred to as chronic myeloid leukemia), therapy related leukemia, MDS, myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, nucleophosmin (NPM1) AML, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia, or malignancies-driven by multiple gene-fusions, rearrangements, or mutations (Issam, G. C. et al. Therapeutic implications of menin inhibitors in acute leukemias, Leukemia 2021, 35, pp. 2482-2495)). In some embodiments, the AML is abstract nucleophosmin (NPM1)-mutated AML (i.e., NPM1^{mut} acute myeloid leukemia),

In particular embodiments, combinations described herein are used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

Acute leukemias generally result from acquired mutations in hematopoietic stem/progenitor cells. Chromosomal abnormalities are often discrete mutational features in leukemia. Many of these chromosomal abnormalities are due to specific translocations that lead to the formation of fusion genes which become drivers for tumorigenesis and tumor development. A specific example involves the MLL1 gene. Translocations at the MLL1 locus (11q23) can lead to the formation of oncogenic gene fusions that characterize MLL-r acute leukemias. The MLL1 protein is a key regulator of development and is the mammalian homologue of Drosophila trithorax. It is an important epigenetic regulator of HOX gene expression. Translocations at the MLL1 locus create chimeric proteins that fuse the N-terminus of MLL1 to variable C-terminal domains derived from different translocation partners. Currently, more than 90 different fusion partners are known. Expression of these fusions enables an aberrant transcription program characterized by overexpression of HOX and other developmental genes. This transcription program suppresses differentiation and enhances proliferation, leading to the MLL-r acute leukemias. Translocations involving the MLL1 locus (11q23) are routinely diagnosed using fluorescence in situ hybridization (FISH). Depending on the progenitor cell of origin, MLL-r can phenotypically appear as ALL, AML, or mixed phenotype acute leukemia (MPAL). These translocations are rare and MLL-r has a combined annual incidence of ˜4000 cases per year in the United States (US), Europe and Japan. Approximately 10% of all leukemias harbor MLL1 translocations.

The present combination is additionally useful for the treatment of leukemia patients with an MLL/KMT2A gene rearrangement.

The relapse risk for MLL-r patients is high after conventional chemotherapy and stem cell transplantation, with an overall 5-year survival rate of only approximately 35%. No therapies are currently available that specifically target MLL-r leukemia. The menin inhibitor (e.g., Compound I or Compound II) in combination with a CYP3A4 inhibitor may provide a novel, targeted treatment for MLL-r acute leukemias.

The interaction of MLL1 fusion proteins with menin is a key driver of MLL-r acute leukemias. Both MLL1 and MLL-r fusions bind to a well-characterized high affinity site on the chromatin-associated protein menin. The binding of MLL1 fusions to menin is mediated by amino acid residues 9-13 (FPARP) found at the N-terminus of MLL1. Binding to menin localizes these fusions to chromatin where they enable a leukemic transcription program, which includes upregulation of HOXA locus and MEIS1 genes. The interaction between the fusion protein and menin is required to maintain this transcription program.

The menin inhibitors Compound (I) and Compound (II) bind with high affinity to the MLL1 binding pocket on menin and displays activity across a range of cells harboring MLL-r fusions. Menin inhibitors Compound (I) and Compound (II) disrupt the interaction between menin and the MLL1 fusion proteins which is required for leukemogenic activity, thus impairing expression of critical oncogenes, causing growth arrest and the inhibition of cellular proliferation. Small molecule inhibitors of the menin-MLL interaction have been reported. These inhibitors have demonstrated anti-proliferative activity against MLL-r cell lines and have shown single agent survival benefit in mouse models of MLL-r leukemia.

Similarly, combining menin inhibitors (e.g., Compound (I) or Compound (II)) II in with a CYP3A4 inhibitor increases efficacy and has demonstrated robust activity in multiple leukemic xenograft models and provided profound survival benefit after oral dosing in nonclinical models. Overall, these data indicate that pharmacologic inhibition of the menin-MLL interaction represents a potential targeted strategy for the treatment MLL-r acute leukemias.

In an aspect, the leukemia is mutated Nucleophosmin 1 (NPM1).

In some embodiments, the combination of the present invention is directed to the treatment of NPM1-mutated leukemia, e.g., AML. The NPM1 gene, encoding for a primarily nucleolar localized multifunctional protein, is the most commonly mutated gene in adult AML (approximately 30% of cases). The mutations (NPM1c) result in their aberrant cytoplasmic localization. Interestingly, the interaction of MLL1 with menin in NPM1c AML shares a common HOX gene signature and dependencies as that of MLL1-r with menin. Indeed, inhibition of menin has demonstrated anti-leukemia activity in both NPM1c and MLL-r AML. NPM1 mutations in AML frequently occur in patients carrying other mutations, such as FLT3. NPM1c cooperates with FLT3-ITD as well as the tyrosine kinase domain (TKD) mutations to promote AML development. Co-inhibition of menin and FLT3 has demonstrated enhanced anti-leukemia activity in MLL-r/FLT3- and NPM1c/FLT3-mutated AML.

In some embodiments, the present invention is directed to the treatment of NPM1 AML in a patient in need thereof comprising administering a menin inhibitor and a CYP3A4 inhibitor. In some further embodiments, the present invention is directed to the treatment of NPM1 AML in a patient in need thereof comprising administering a pharmaceutical composition comprising a menin inhibitor and a pharmaceutical composition comprising a CYP3A4 inhibitor. In some further embodiments, the present invention is directed to the treatment of NPM1 AML in a patient in need thereof comprising administering a pharmaceutical composition comprising a menin inhibitor (e.g., Compound (I) or Compound (II)) and a pharmaceutical composition comprising an azole antifungal CYP3A4 inhibitor.

In a further aspect, the cancer with or without MLL-r and with or without NPM1 mutations can also have FLT3 mutations. Mutations in FLT3 are diagnosed in about one third of newly diagnosed AML patients, for example. FLT3 internal tandem duplications are associated with increased relapse and poor overall survival.

Targeting Bcl-2, a critical survival factor for AML, has emerged as a promising therapeutic option for patients with AML. However, despite a major increase in CR/CRi's by combining the Bcl-2 inhibitor venetoclax with hypomethylating agents, most patients develop resistance and ultimately relapse. As Bcl-2 is a pan anti-apoptotic protein and its inhibition lowers apoptotic threshold, venetoclax has become a mainstay for combinatorial therapies.

In some embodiments, a subject treated in accordance with the methods described herein has previously been treated with a Bcl-2 inhibitor. In some embodiments, a subject treated in accordance with the methods described herein has previously been treated with a Bcl-2 inhibitor and has developed resistance to the Bel-2 inhibitor. In some embodiments, a subject treated in accordance with the method described herein has previously been treated with a Bcl-2 inhibitor for a cancer and the cancer progressed on the prior Bcl-2 inhibitor treatment.

In some embodiments, a subject treated in accordance with the methods described herein has previously been treated with venetoclax. In some embodiments, a subject treated in accordance with the methods described herein has previously been treated with venetoclax and has developed resistance to venetoclax. In some embodiments, a subject treated in accordance with the method described herein has previously been treated with venetoclax for a cancer and the cancer progressed on the prior venetoclax treatment.

The efficacy of a method of treatment described herein may be evaluated using any suitable method known in the art or described herein. In some embodiments, the efficacy of a method of treatment described herein is evaluated by measuring the number of leukemia cells (e.g., human CD45⁺ cells) in the blood, the spleen, or the bone marrow of the subject using flow cytometry. In some embodiments, the efficacy of a method of treatment described herein is evaluated by determining the size of the spleen of a subject. Many patients treated with venetoclax/hypomethylating agents ultimately progress or develop resistance to venetoclax-based therapies. However, the inventors have found that MV4-11 cells (with MLL-r and FLT3-ITD) with acquired resistance to venetoclax are sensitive to a menin inhibitor such as Compound I.

In some embodiments, the combination of the present disclosure exhibited strong anti-leukemia activity and significantly prolonged survival, while venetoclax alone had minimal effect. In some embodiments, the menin inhibition preferentially targeted CD34+CD38+ cells. In some embodiments, venetoclax targeted CD34+CD38− cells. In some embodiments, the combined inhibition of menin and Bel-2 effectively eliminated bulk and CD34+CD38+/CD34+CD38− stem/progenitor cells. In some embodiments, the administration of the combination increased the CD11b+ myeloid cell population. In some embodiments, the administration of the combination of a menin inhibitor and venetoclax synergistically increased the CDb11+ myeloid cell population. In an aspect, the combination of the therapeutically effective amount of the menin inhibitor and the therapeutically effective amount of a Bcl-2 inhibitor synergistically reduces leukemia CD34⁺CD38⁺/CD34⁺CD38⁻ stem/progenitor cells in bone marrow.

In some embodiments, the efficacy of a method of treatment described herein is determined by measuring the expression of pro-apoptotic proteins (e.g., Bim) in a subject (e.g., in CD34⁺CD38⁺ cells of a subject). In some embodiments, the efficacy of a method of treatment described herein is determined by measuring the expression of anti-apoptotic proteins (e.g., Bcl-2 and/or Bcl-xL) in a subject (e.g., in CD34⁺CD38⁺ cells of a subject). In some embodiments, the efficacy of a method of treatment described herein is determined by measuring the expression of proteins associated with resistance to treatment with Bcl-2 inhibitors (e.g., Bcl-2A1) in a subject (e.g., in human CD45 cells of a subject). The expression of proteins may be determined using any suitable method known in the art or described herein including, for example, flow cytometry, immunohistochemistry, or Western Blotting. Suitable samples in which protein expression can be analyzed include, without limitation, the blood, bone marrow and the spleen.

In some embodiments, the efficacy of a method of treatment described herein is evaluated by measuring overall survival and/or progression-free survival of a subject at a suitable time point (e.g., 1 month, 2 months, 3 months, 6 months, 9 months, 12 months, 18 months, 2 years, 3 years, 4 years, 5 years, 10 year, or 15 years) after treatment.

Treating cancer can result in a reduction in size of a tumor. A reduction in size of a tumor may also be referred to as “tumor regression”. Preferably, after treatment, tumor size is reduced by 5%, 10%, 20%, 30%, 40%, 50%, or 75% or greater relative to its size prior to treatment. Size of a tumor may be measured by any reproducible means of measurement. The size of a tumor may be measured as a diameter of the tumor.

Treating cancer in accordance with a method described herein can result in a reduction in tumor volume. Preferably, after treatment, tumor volume is reduced by 5%, 10%, 20%, 30%, 40%, 50%, or 75% or greater. Tumor volume may be measured by any reproducible means of measurement.

Treating cancer in accordance with a method described herein can result in a decrease in number of tumors. Preferably, after treatment, tumor number is reduced by 5%, 10%, 20%, 30%, 40%, 50%, or 75% or greater. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer in accordance with a method described herein can result in a decrease in number of metastatic lesions in other tissues or organs distant from the primary tumor site. Preferably, after treatment, the number of metastatic lesions is reduced by 5%, 10%, 20%, 30%, 40%, 50%, or 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer in accordance with a method described herein can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer in accordance with a method described herein can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer in accordance with a method described herein can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer in accordance with a method described herein can result in a decrease in the mortality rate of a population of treated subjects in comparison to a population receiving carrier alone. Treating cancer can result in a decrease in the mortality rate of a population of treated subjects in comparison to an untreated population. Treating cancer in accordance with a method described herein can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer in accordance with a method described herein can result in a decrease in tumor growth rate. Preferably, after treatment, tumor growth rate is reduced by at least 5%, 10%, 20%, 30%, 40%, 50%, or 75%. Tumor growth rate may be measured by any reproducible means of measurement. Tumor growth rate can be measured according to a change in tumor diameter per unit time.

Treating cancer in accordance with a method described herein can result in a decrease in tumor regrowth. Preferably, after treatment, tumor regrowth is less than 5%, 10%, 20%, 30%, 40%, 50%, or 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer or a cell proliferative disorder in accordance with a method described herein can result in cell death, and preferably, cell death results in a decrease of at least 10% in number of cells in a population. More preferably, cell death means a decrease of at least 10%, 20%, 30%, 40%, 50%, or 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

The therapeutic combinations provided herein can result in a synergistic effect in the treatment of a disease or cancer. A “synergistic effect” is defined as where the efficacy of a combination of the agents of a therapeutic combination (e.g., a menin inhibitor and a Bcl-2 inhibitor) is greater than the sum of the effects of any of the agents given alone. A synergistic effect may also be an effect that cannot be achieved by administration of any of the agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

As provided herein, treatment with a therapeutic combination provided herein result in a synergistic antiproliferative response, a synergistic induction of apoptosis in leukemic cells, a synergistic induction of differentiation of leukemic cells, and a synergistic extension of survival.

### Combination Therapies

As provided herein, “combination therapy” also embraces the administration of the therapeutic combinations described herein in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic combination and non-drug treatment is achieved. For example, in appropriate cases, the beneficial effect is still achieved when the non-drug treatment is temporally removed from the administration of the therapeutic combination, perhaps by days or even weeks.

In another aspect, a therapeutic combination of the present invention may be administered in combination with radiation therapy. Radiation therapy can also be administered in combination with a composition of the present invention and another chemotherapeutic agent described herein as part of a multiple agent therapy.

In certain instances, it is appropriate to administer a therapeutic combination comprising a menin inhibitor and a Bcl-2 inhibitor (and optionally a CYP3A4 inhibitor, a hypomethylating agent, an FLT3 inhibitor, or a combination thereof) provided herein in combination with an additional therapeutic agent.

Additional therapeutic agents may be selected for their particular usefulness against the condition that is being treated. In general, the additional therapeutic agent does not need to be administered in the same pharmaceutical composition, at the same time or via the same route as the therapeutic combination comprising a menin inhibitor and a Bcl-2 inhibitor (and optionally a CYP3A4 inhibitor, a hypomethylating agent, an FLT3 inhibitor, or both) provided herein. In some embodiments, the initial administration of the additional therapeutic agent is made according to established protocols, and then, based upon the observed effects, the dosage, modes of administration and times of administration are further modified.

In some embodiments, the additional therapeutic agent is administered concurrently (e.g., simultaneously, essentially simultaneously or within the same treatment protocol) or sequentially, depending upon the nature of the disease, the condition of the patient, and the actual choice of compounds used. In certain embodiments, the determination of the order of administration, and the number of repetitions of administration of each therapeutic agent during a treatment protocol, is based upon evaluation of the disease being treated and the condition of the patient.

The dose of the additional therapeutic agent varies depending on the additional therapeutic agent, the disease or condition being treated and so forth.

In some embodiments, the additional therapeutic agent is a chemotherapeutic agent, a steroid, an immunotherapeutic agent, a targeted therapy, or a combination thereof. In some embodiments, the additional therapeutic agent is a CD79A inhibitor, a CD79B inhibitor, a CD 19 inhibitor, a Lyn inhibitor, a Syk inhibitor, a PI3K inhibitor, a Bank inhibitor, a PLCy inhibitor, a PKCP inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak12 inhibitor, a protease inhibitor, a PKC inhibitor, a PARP inhibitor, or a combination thereof.

In some embodiments, the additional therapeutic agent is chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

In some embodiments, the additional therapeutic agent is cyclophosphamide, hydroxydaunorubicin, vincristine, and prednisone, and optionally, rituximab. In some embodiments, the additional therapeutic agent is bendamustine, and rituximab. In some embodiments, the additional therapeutic agent is fludarabine, cyclophosphamide, and rituximab. In some embodiments, the additional therapeutic agent is cyclophosphamide, vincristine, and prednisone, and optionally, rituximab. In some embodiments, the additional therapeutic agent is ctoposide, doxorubicin, vincristine, cyclophosphamide, prednisolone, and optionally, rituximab. In some embodiments, the additional therapeutic agent is dexamethasone and lenalidomide.

Additional therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to, a Nitrogen Mustard such as for example, bendamustine, chlorambucil, chlormethine, cyclophosphamide, ifosfamide, melphalan, prednimustine, trofosfamide; an Alkyl Sulfonate such as for example busulfan, mannosulfan, treosulfan; an Ethylene Imine such as, for example carboquone, thiotepa, triaziquone; a Nitrosourea such as for example carmustine, fotemustine, lomustine, nimustine, ranimustine, semustine, streptozocin; an Epoxide such as for example, etoglucid; another Alkylating Agent such as for example dacarbazine, mitobronitol, pipobroman, temozolomide; a Folic Acid Analog such as for example methotrexate, permetrexed, pralatrexate, raltitrexed; a Purine Analog such as for example cladribine, clofarabine, fludarabine, mercaptopurine, nelarabine, tioguanine; a Pyrimidine Analog such as for example azacitidine, capecitabine, carmofur, cytarabine, decitabine, fluorouracil, gemcitabine, tegafur; a Vinca Alkaloid such as for example vinblastine, vincristine, vindesine, vinflunine, vinorelbine; a Podophyllotoxin Derivative such as for example etoposide, teniposide; a Colchicine derivative such as for example demecolcine; a Taxane such as for example docetaxel, paclitaxel, paclitaxel poliglumex; another plant alkaloid or a natural product such as for example trabectedin; an Actinomycine such as for example dactinomycin; an Antracycline such as for example aclarubicin, daunorubicin, doxorubicin, epirubicin, idarubicin, mitoxantrone, pirarubicin, valrubicin, zorubincin; another Cytotoxic Antibiotic such as for example bleomycin, ixabepilone, mitomycin, plicamycin; a Platinum Compound such as for example carboplatin, cisplatin, oxaliplatin, satraplatin; a Methylhydrazine such as for example procarbazine; a Sensitizer such as for example aminolevulinic acid, efaproxiral, methyl aminolevulinate, porfimer sodium, temoporfm; a Protein Kinase Inhibitor such as for example dasatinib, erlotinib, everolimus, gefitinib, imatinib, lapatinib, nilotinib, pazonanib, sorafenib, sunitinib, temsirolimus; another Antineoplastic Agent such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; an Estrogen such as for example diethylstilbenol, ethinylestradiol, fosfestrol, polyestradiol phosphate; A Progestogen such as for example gestonorone, medroxyprogesterone, megestrol; a Gonadotropin Releasing Hormone Analog such as for example buserelin, goserelin, leuprorelin, triptorelin; an Anti-Estrogen such as for example fulvestrant, tamoxifen, toremifene; an Anti-Androgen such as for example bicalutamide, flutamide, nilutamide, an Enzyme Inhibitor such as for example aminoglutethimide, anastrozole, exemestane, formestane, letrozole, vorozole; another Hormone Antagonist such as for example abarelix, degarelix; an Immunostimulant such as for example histamine dihydrochloride, mifamurtide, pidotimod, plerixafor, roquinimex, thymopentin; an Immunosuppressant such as for example everolimus, gusperimus, leflunomide, mycophenolic acid, sirolimus; a Calcineurin Inhibitor such as for example ciclosporin, tacrolimus; another Immunosuppressant such as for example azathioprine, lenalidomide, methotrexate, thalidomide; and a Radiopharmaceutical such as for example, iobenguane.

Further therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to an interferon, an interleukin, a Tumor Necrosis Factor, and a Growth Factor.

Additional therapeutic agents that maybe administered in conjunction a therapeutic combination comprising a menin inhibitor and a Bcl-2 inhibitor (and optionally a CYP3A4, an FLT3 inhibitor, or both) provided herein include, but are not limited to, an immunostimulant such as for example ancestim, filgrastim, lenograstim, molgramostim, pegfilgrastim, sargramostim; an Interferon such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; an Interleukin such as for example aldesleukin, oprelvekin; another Immunostimulant such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; an Immunosuppressant such as for example abatacept, abetimus, alefacept, antilymphocyte immunoglobulin (horse), antithymocyte immunoglobulin (rabbit), cculizumab, efalizumab, everolimus, gusperimus, leflunomide, muromab-CD3, mycophenolic acid, natalizumab, sirolimus; a TNF alpha Inhibitor such as for example adalimumab, afelimomab, certolizumab pegol, etanercept, golimumab, infliximab; an Interleukin Inhibitor such as for example anakinra, basiliximab, canakinumab, daclizumab, mepolizumab, rilonacept, tocilizumab, ustekinumab; a Calcineurin Inhibitor such as for example ciclosporin, tacrolimus; another Immunosuppressant such as for example azathioprine, lenalidomide, methotrexate, thalidomide.

Further therapeutic agents that maybe administered in conjunction a therapeutic combination comprising a menin inhibitor and a Bcl-2 inhibitor (and optionally a CYP3A4, an FLT3 inhibitor, or both) provided herein include, but are not limited to, Adalimumab, Alemtuzumab, Basiliximab, Bevacizumab, Cetuximab, Certolizumab pegol, Daclizumab, Eculizumab, Efalizumab, Gemtuzumab, Ibritumomab tiuxetan, Infliximab, Muromonab-CD3, Natalizumab, Panitumumab, Ranibizumab, Rituximab, Tositumomab, Trastuzumabor a combination thereof.

Additional therapeutic agents that maybe administered in conjunction a therapeutic combination comprising a menin inhibitor and a Bcl-2 inhibitor provided (and optionally a CYP3A4, an FLT3 inhibitor, or both) herein include, but are not limited to, a Monoclonal Antibody such as for example alemtuzumab, bevacizumab, catumaxomab, cetuximab, cdrecolomab, gemtuzumab, ofatumumab, panitumumab, rituximab, trastuzumab, an Immunosuppressant such as for example, eculizumab, efalizumab, muromab-CD3, natalizumab; a TNF alpha Inhibitor such as for example adalimumab, afelimomab, certolizumab pegol, golimumab, infliximab, an Interleukin Inhibitor, basiliximab, canakinumab, daclizumab, mepolizumab, tocilizumab, ustekinumab, a Radiopharmaceutical, ibritumomab tiuxetan, tositumomab; another Monoclonal Antibody such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, ctaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

Further therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to, an agent that affects the tumor micro-environment such as cellular signaling network (e.g. phosphatidylinositol 3-kinase (PI3K) signaling pathway, signaling from the B-cell receptor and the IgE receptor). In some embodiments, the second agent is a PI3K signaling inhibitor or a syc kinase inhibitor. In some embodiments, the syk inhibitor is R788. In another embodiment, the second agent is a PKCy inhibitor such as by way of example only, enzastaurin.

Examples of agents that affect the tumor micro-environment include a PI3K signaling inhibitor, a syc kinase inhibitor, a Protein Kinase Inhibitor such as for example dasatinib, erlotinib, everolimus, gefitinib, imatinib, lapatinib, nilotinib, pazonanib, sorafenib, sunitinib, temsirolimus; another Angiogenesis Inhibitor such as for example GT-111, JI-101, R1530; another Kinase Inhibitor such as for example AC220, AC480, ACE-041, AMG 900, AP24534, Arry-614, AT7519, AT9283, AV-951, axitinib, AZD1152, AZD7762, AZD8055, AZD8931, bafctinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, SCH 727965, SGI-1176, SGX523, SNS-314, TAK-593, TAK-901, TKI258, TLN-232, TTP607, XL147, XL228, XL281R05126766, XL418, XL765.

Further examples of therapeutic agents for use in combination with a therapeutic combination comprising a menin inhibitor and a Bcl-2 inhibitor (and optionally a CYP3A4, an FLT3 inhibitor, or both) provided herein include, but are not limited to, an inhibitor of mitogen-activated protein kinase signaling, e.g., U0126, PD98059, PD184352, PD0325901, ARRY-142886, SB239063, SP600125, BAY 43-9006, wortmannin, or LY294002; a Syk inhibitor; an mTOR inhibitor; and an antibody (e.g., rituxan).

Other agents that may be employed in combination with a therapeutic combination comprising a menin inhibitor and a Bcl-2 inhibitor (and optionally a CYP3A4, an FLT3 inhibitor, or both) provided herein include, but are not limited to, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol; chlorambucil; cirolemycin; cladribine; crisnatol mesylate; cyclophosphamide; cytarabinc; dacarbazine; daunorubicin hydrochloride; decitabine; dexormap latin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; ctoposide; ctoposide phosphate; etoprine; fadrozole hydrochloride; fazarabine; fenretinide; floxuridine; fiudarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rIL2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safmgol; safmgol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfm; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to, 20-epi-1, 25 dihydroxyvitamin D3; 5-ethynyluracil; abiraterone; aclarubicin; acylfulvene; adecypenol; adozelesin; aldesleukin; an ALL-TK antagonist; altretamine; ambamustine; amidox; amifostine; aminolevulinic acid; amrubicin; amsacrine; anagrelide; anastrozole; andrographolide; an angiogenesis inhibitor; antagonist D; antagonist G; antarelix; anti-dorsalizing morphogenetic protein-1; antiandrogen, prostatic carcinoma; antiestrogen; antineoplaston; an antisense oligonucleotide; aphidicolin glycinate; an apoptosis gene modulator; an apoptosis regulator; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; a BCR/ABL antagonist; benzochlorins; benzoylstaurosporine; a beta lactam derivative; beta-alethine; betaclamycin B; betulinic acid; a bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; a camptothecin derivative; canarypox IL-2; capecitabine; carboxamide-amino-triazole; carboxyamidotriazole; CaRest M3; CARN 700; a cartilage derived inhibitor; carzelesin; a casein kinase inhibitor (ICOS); castanospermine; cecropin B; cetrorelix; chlorlns; chloroquinoxaline sulfonamide; cicaprost; cis-porphyrin; cladribine; a clomifene analog; clotrimazole; collismycin A; collismycin B; combretastatin A4; a combretastatin analog; conagenin; crambescidin 816; crisnatol; cryptophycin 8; a cryptophycin A derivative; curacin A; cyclopentanthraquinones; cycloplatam; cypemycin; cytarabine ocfosfate; a cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; an estramustine analog; an estrogen agonist; an estrogen antagonist; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; a gelatinase inhibitor; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; an immunostimulant peptide; an insulin-receptor inhibitor; an interferon agonist; an interferon; an interleukin; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; a linear polyamine analogue; a lipophilic disaccharide peptide; a lipophilic platinum compound; lissoclinamide 7; lobaplatin; lombricine; lometrexol; lonidamine; losoxantrone; lovastatin; loxoribine; lurtotecan; lutetium texaphyrin; lysofylline; a lytic peptides maitansine; mannostatin A; marimastat; masoprocol; maspin; matrilysin inhibitors; a matrix metalloproteinase inhibitor; menogaril; merbarone; meterelin; methioninase; metoclopramide; a MIF inhibitor; mifepristone; miltefosine; mirimostim; mismatched double stranded RNA; mitoguazone; mitolactol; a mitomycin analog; mitonafide; mitotoxin fibroblast growth factor-saporin; mitoxantrone; mofarotene; molgramostim;, human chorionic gonadotrophin; monophosphoryl lipid A+myobacterium cell wall sk; mopidamol; a multiple drug resistance gene inhibitor; a multiple tumor suppressor 1-based therapy; a mustard anticancer agent; mycaperoxide B; mycobacterial cell wall extract; myriaporone; N-acetyldinaline; an N-substituted benzamide; nafarelin; nagrestip; naloxone+pentazocine; napavin; naphterpin; nartograstim; nedaplatin; nemorubicin; neridronic acid; neutral endopeptidase; nilutamide; nisamycin; a nitric oxide modulator; an nitroxide antioxidant; nitrullyn; 06-benzylguanine; octreotide; okicenone; an oligonucleotide; onapristone; ondansetron; ondansetron; oracin; an oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; a phosphatase inhibitor; picibanil; pilocarpine hydrochloride; pirarubicin; piritrexim; placetin A; placetin B; a plasminogen activator inhibitor; a platinum complex; a platinum compound; platinum-triamine complex; porfimer sodium; porfiromycin; prednisone; propyl bis-acridone; prostaglandin J2; a proteasome inhibitor; protein A-based immune modulator; a protein kinase C inhibitor; protein kinase C inhibitors, microalgal; a protein tyrosine phosphatase inhibitor; a purine nucleoside phosphorylase inhibitor; a purpurin; pyrazoloacridine; pyridoxylated hemoglobin polyoxyethyleric conjugate; a raf antagonist; raltitrexed; ramosetron; ras a farnesyl protein transferase inhibitor; a ras inhibitor; a ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safmgol; saintopin; SarCNU; sarcophytol A; sargramostim; an Sdi 1 mimetic; semustine; senescence derived inhibitor 1; sense oligonucleotides; a signal transduction inhibitor; a signal transduction modulator; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; a stem cell inhibitor; a stem-cell division inhibitor; stipiamide; a stromelysin inhibitor; sulfinosine; superactive vasoactive intestinal peptide antagonist; suradista; suramin; swainsonine; a synthetic glycosaminoglycan; tallimustine; tamoxifen methiodide; tauromustine; tazarotene; tecogalan sodium; tegafur; tellurapyrylium; a telomerase inhibitor; temoporfm; temozolomide; teniposide; tetrachlorodecaoxide; tetrazomine; thaliblastine; thiocoraline; thrombopoietin; a thrombopoietin mimetic; thymalfasin; a thymopoietin receptor agonist; thymotrinan; thyroid stimulating hormone; tin ethyl ctiopurpurin; tirapazamine; titanocene bichloride; topsentin; toremifene; totipotent stem cell factor; a translation inhibitor; tretinoin; triacetyluridine; triciribine; trimetrexate; triptorelin; tropisetron; turosteride; a tyrosine kinase inhibitor; tyrphostins; a UBC inhibitor; ubenimex; urogenital sinus-derived growth inhibitory factor; a urokinase receptor antagonist; vapreotide; variolin B; vector system, erythrocyte gene therapy; velaresol; veramine; verdins; verteporfm; vinorelbine; vinxaltine; vitaxin; vorozole; zanoterone; zeniplatin; zilascorb; and zinostatin stimalamer.

Other therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to, another CYP3A4 inhibitor, an alkylating agent, an antimetabolite, a natural product or hormone, a nitrogen mustard (e.g., mechloroethamine, cyclophosphamide, chlorambucil, etc.), an alkyl sulfonate (e.g., busulfan), a nitrosourea (e.g., carmustine, lomusitne, etc.), or triazenes (decarbazine, etc.). Examples of antimetabolites include but are not limited to folic acid analogs (e.g., methotrexate), or pyrimidine analogs (e.g., Cytarabine), purine analogs (e.g., mercaptopurine, thioguanine, pentostatin).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to, folic acid analog (e.g., methotrexate), or pyrimidine analogs (e.g., fluorouracil, floxouridine, Cytarabine), purine analogs (e.g., mercaptopurine, thioguanine, pentostatin.

Additional therapeutic agents that maybe administered in conjunction with a therapeutic combination comprising a menin inhibitor and a Bcl-2 inhibitor (and optionally a CYP3A4, an FLT3 inhibitor, or both) provided herein include, but are not limited to,: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), an Epothilone (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminocpothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCI), AC-7700 (Ajinomoto, also known as AVE-8062, AVE-8062A, CS-39-L-Ser.HCI, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribacoside, Caribacolin, Halichondrin B, D-64131 (Asta Medica), D-68144 (Asta Medica), Diazonamide A, A-293620 (Abbott), NPI-2350 (Nereus), Taccalonolide A. TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifiuoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

A therapeutic combination comprising a menin inhibitor and a Bcl-2 inhibitor (and optionally a CYP3A4, an FLT3 inhibitor, or both) provided herein may be used in combination with: an immunosuppressant (e.g., tacrolimus, cyclosporin, rapamicin, methotrexate, cyclophosphamide, azathioprine, mercaptopurine, mycophenolate, or FTY720), a glucocorticoid (e.g., prednisone, cortisone acetate, prednisolone, methylprednisolone, dexamethasone, betamethasone, triamcinolone, beclometasone, fludrocortisone acetate, deoxycorticosterone acetate, aldosterone), a non-steroidal anti-inflammatory drug (e.g., salicylates, arylalkanoic acids, 2-arylpropionic acids, N-arylanthranilic acids, oxicams, coxibs, or sulphonanilides), a Cox-2-specific inhibitor (e.g., valdecoxib, celecoxib, or rofecoxib), leflunomide, gold thioglucose, gold thiomalate, aurofm, sulfasalazine, hydroxychloroquinine, minocycline, a TNF-α binding protein (e.g., infliximab, etanercept, or adalimumab), abatacept, anakinra, interferon-β, interferon-γ, interleukin-2, an allergy vaccine, an antihistamine, an antileukotriene, a beta-agonists theophylline, and/or anticholinergics.

### Kits and Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include the menin inhibitor and the Bcl-2 inhibitor and optionally the CYP3A4 inhibitor, the hypomethylating agent, the FLT3 inhibitor, or a combination thereof as disclosed herein. The menin inhibitor and the Bcl-2 inhibitor, the CYP3A4 inhibitor, the hypomethylating agent and/or the FLT3 inhibitor may be provided in one, two, three, or four containers. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

In certain embodiments, pharmaceutical compositions (e.g., a pharmaceutical composition provided herein) are presented in a pack or dispenser device which contains one or more unit dosage forms containing a compound provided herein. The pack, for example, contains metal or plastic foil, such as a blister pack. In some embodiments, the pack or dispenser device is accompanied by instructions for administration. In some embodiments, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are also prepared, placed in an appropriate container, and labeled for treatment of an indicated condition.

### EXAMPLES

The example in this section is provided for illustration only and is not intended to be limiting.

### Example 1. Menin Inhibitor (Compound (I); SNDX-50469) in Combination with Venetoclax

The anti-leukemic activity and potential synergism and mechanisms of the combination of the menin-MLL1 inhibitor Compound (I), an equipotent surrogate of Compound (II), and venetoclax were investigated in vivo in an NPM1c/FLT3-ITD/TKD patient-derived xenograft (PDX) model.

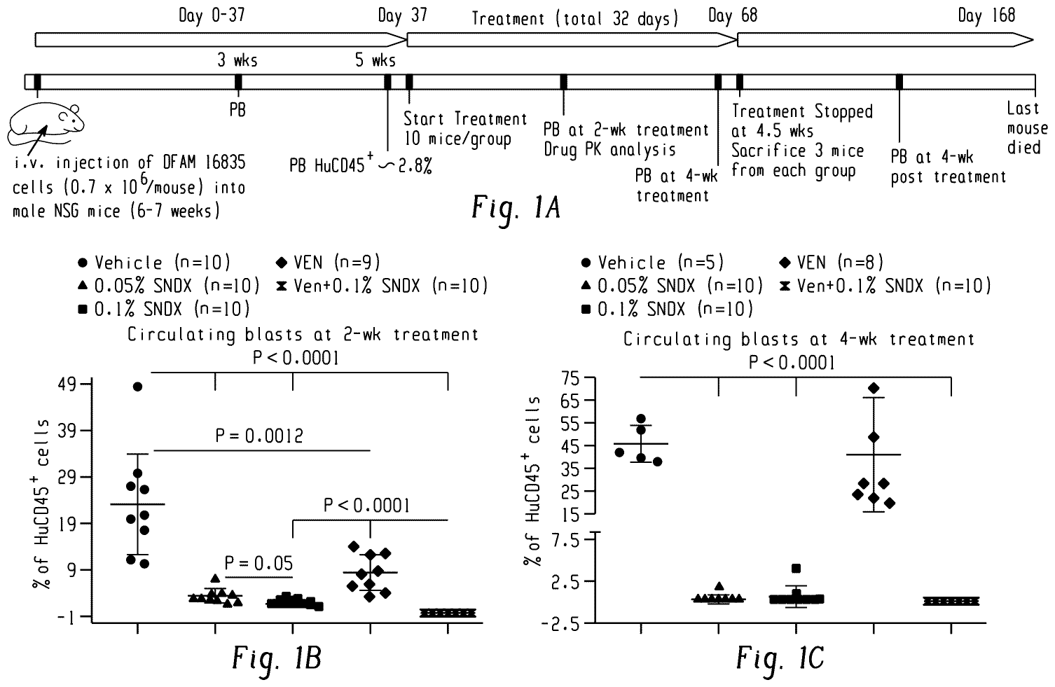

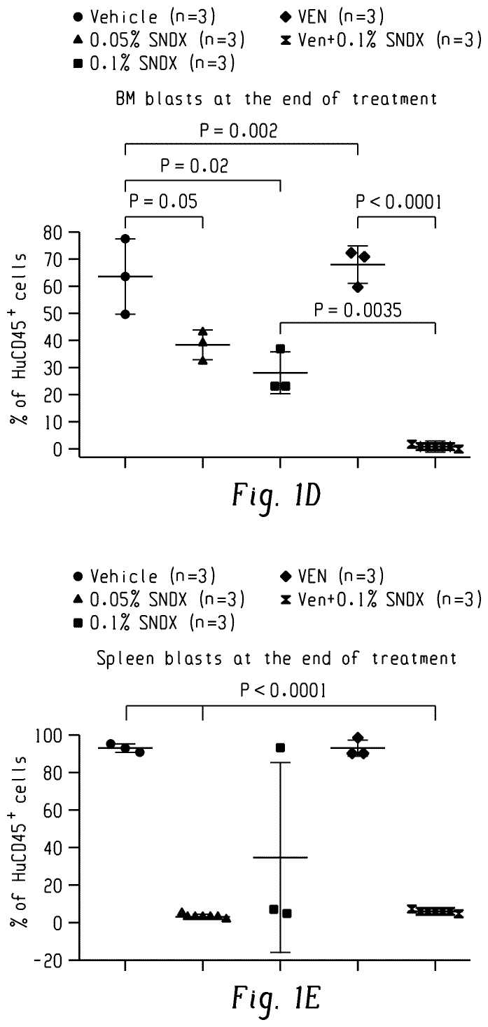

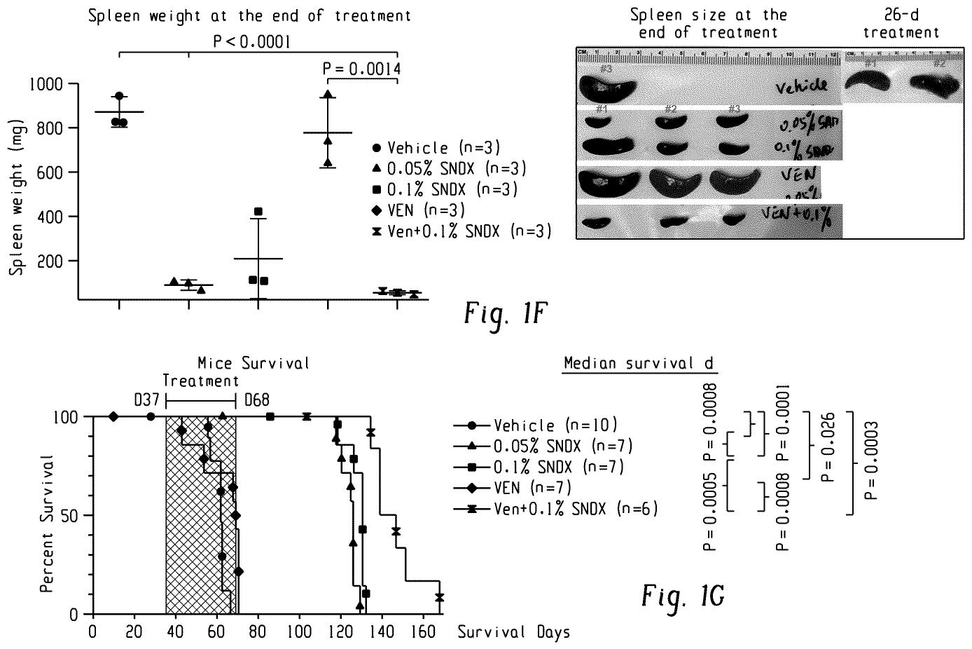

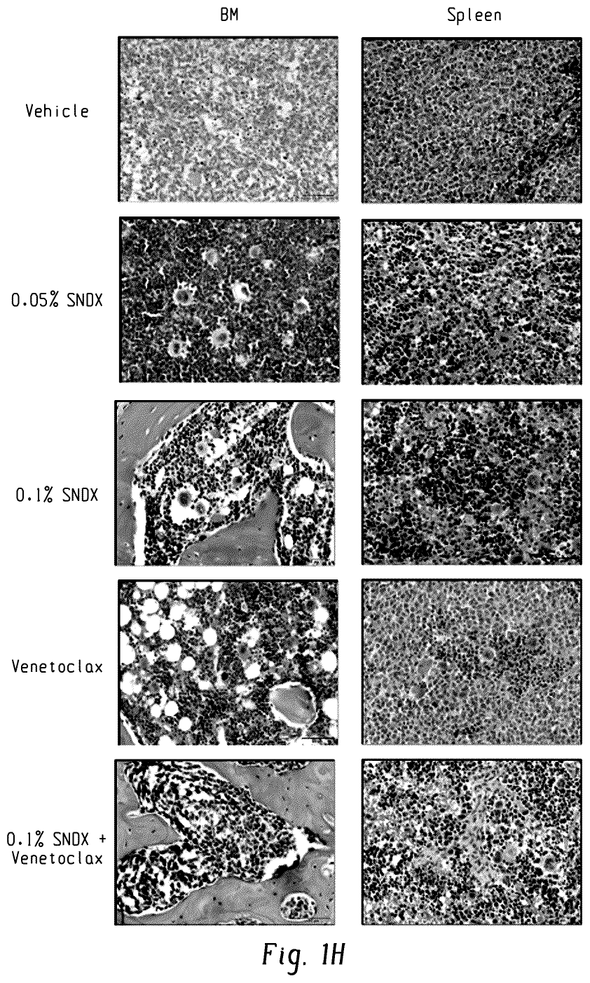

Mouse experiments were performed following institutional animal care and use committee approved protocols. Mouse survival was estimated using the Kaplan-Meier method and survival data were analyzed using the log-rank test. Differences between groups were determined using the Student t-text; P values≤0.05 were considered statistically significant. The PDX (DFAM-16835) was obtained from the PRoXe depository. The engrafted NSG mice were treated with 0.05 or 0.1% Compound (I) (SNDX)-spiked chow, venetoclax (VEN), or 0.1% Compound (I) plus venetoclax (FIG. 1A). At 2 weeks, Compound (I) at either 0.05 or 0.1% (P<0.0001) or venetoclax (P=0.0012) significantly decreased circulating blasts as assessed by flow cytometric measurement of human CD45⁺(huCD45⁺) cells. The higher dose was more effective in this regard (P=0.05), and the combination was significantly more effective than 0.1% Compound (I) or venetoclax (P<0.0001) (FIG. 1B). At 4 weeks, Compound (I), and its combination with venetoclax, significantly (P<0.0001) diminished circulating leukemia cells, while venetoclax alone was ineffective (FIG. 1C).

Flow cytometric analysis revealed that at the end of the treatment, Compound (I) at 0.05% (P=0.05) or 0.1% (P=0.02) partially decreased BM leukemia cells. Although the higher dose tended to be more effective, no statistical significance was reached. Venetoclax alone showed no activity, but it markedly diminished BM leukemia burden when combined with 0.1% Compound I (P=0.0035 vs. 0.1% Compound I) (FIG. 1D). Venetoclax alone also lacked activity in the spleen, while Compound I alone or its combination with venetoclax largely reduced splenic huCD45⁺ cells (FIG. 1E) and spleen weight or size (FIG. 1F) except for one 0.1% Compound I-treated mouse that showed high blasts and an enlarged spleen. This same mouse also showed relatively higher BM huCD45⁺ cells (FIG. 1D). The results were consistent with H&E staining (FIG. 1H).

As shown in FIGS. 1A-1H, menin inhibition demonstrated anti-leukemia activities and prolonged mouse survival which was further enhanced by Bcl-2 inhibition in an NPM1c/FLT3-ITD/TKD PDX model. Thus, Compound (I) at 0.05% or 0.1% significantly extended mouse survival (median 125 and 131 days compared to 61 days of controls, respectively; P=0.0001) and the higher dose showed increased benefit (P=0.008). Venetoclax alone minimally prolonged survival (median 69 d, P=0.026) versus controls. However, mice treated with 0.1% Compound (I) plus venetoclax more than doubled their survival (median 143 d) compared to untreated (P=0.0003) or venetoclax (P=0.0008) treated mice, and further extended survival beyond the 0.1% Compound (I) treated mice (P=0.0005).

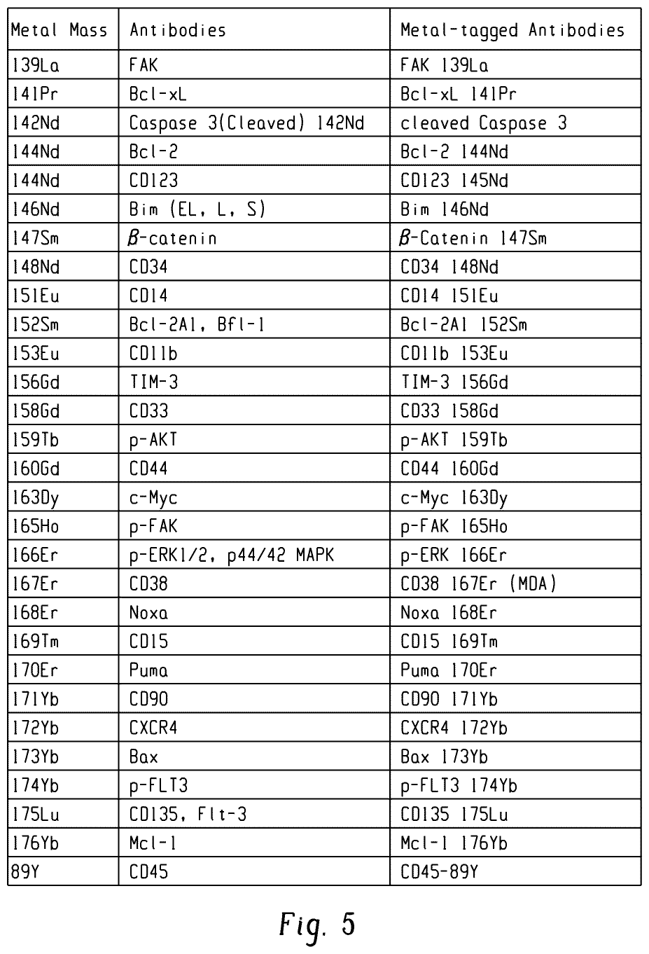

At the end of the therapy, the treatment effects on leukemia blasts and phenotypically-defined leukemia stem/progenitor cells, along with protein expression of BM leukemia cells, were assessed by CyTOF analysis by methods known in the art. The CyTOF panel is shown in FIG. 5.

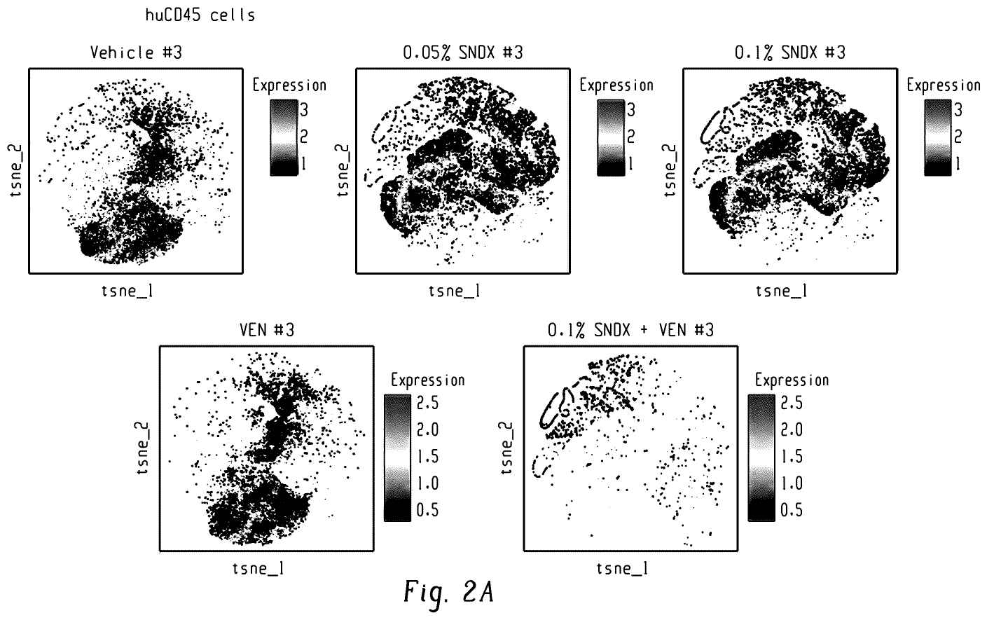

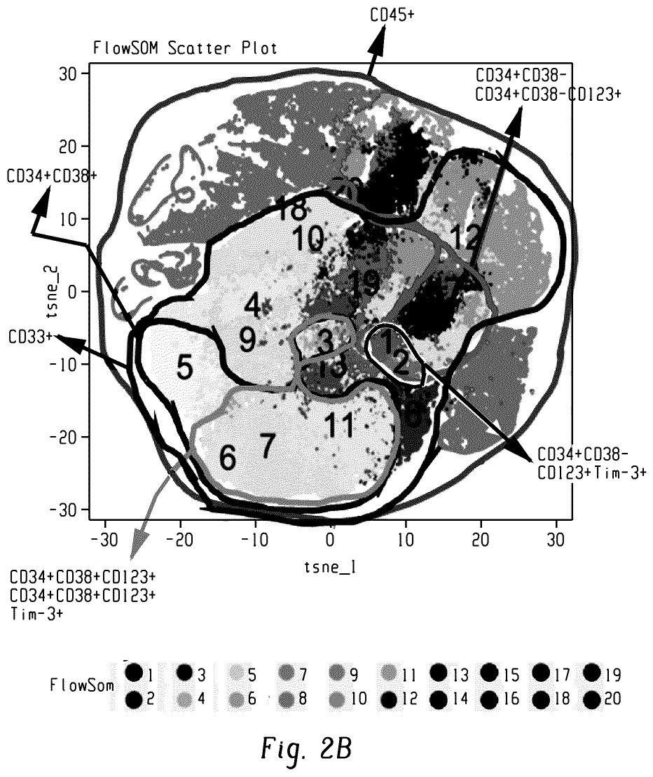

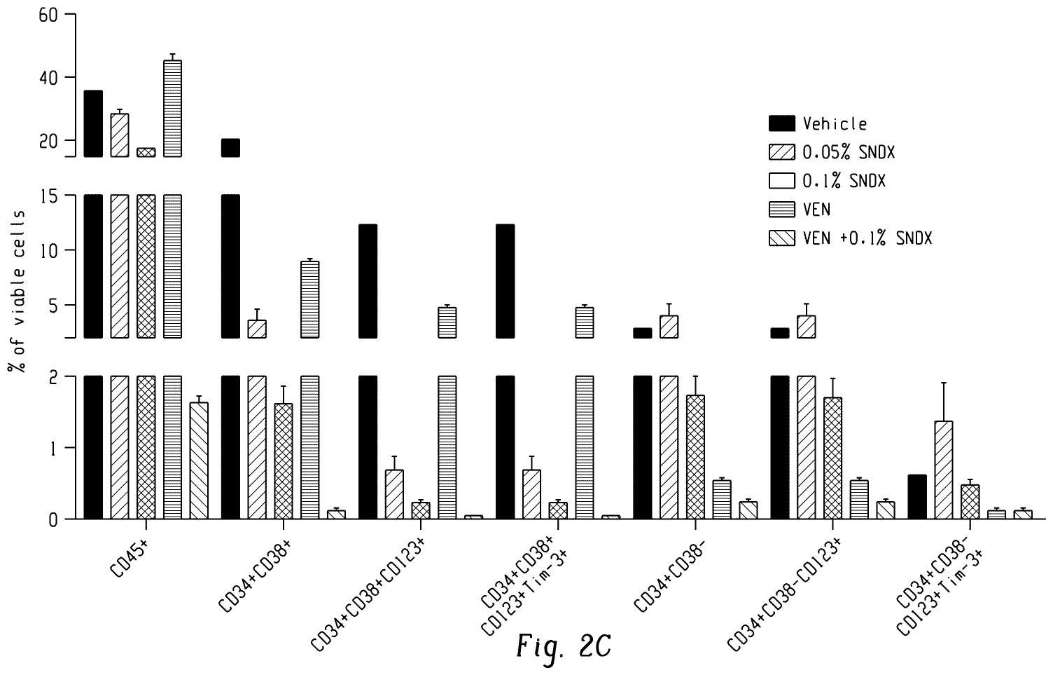

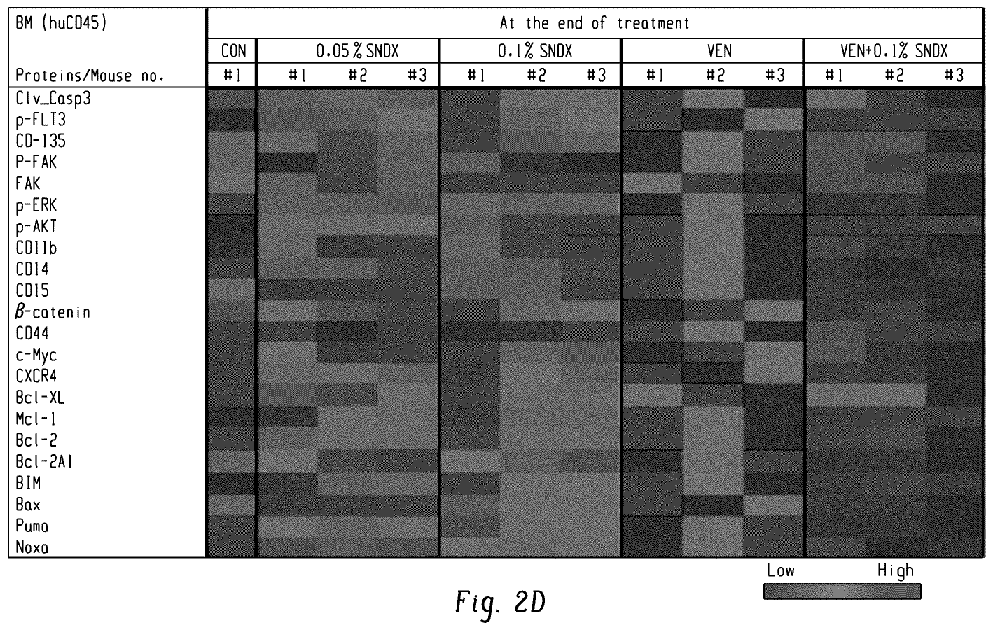

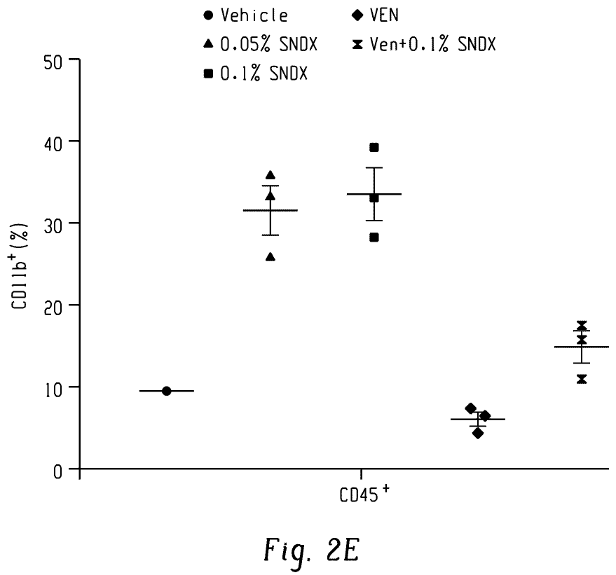

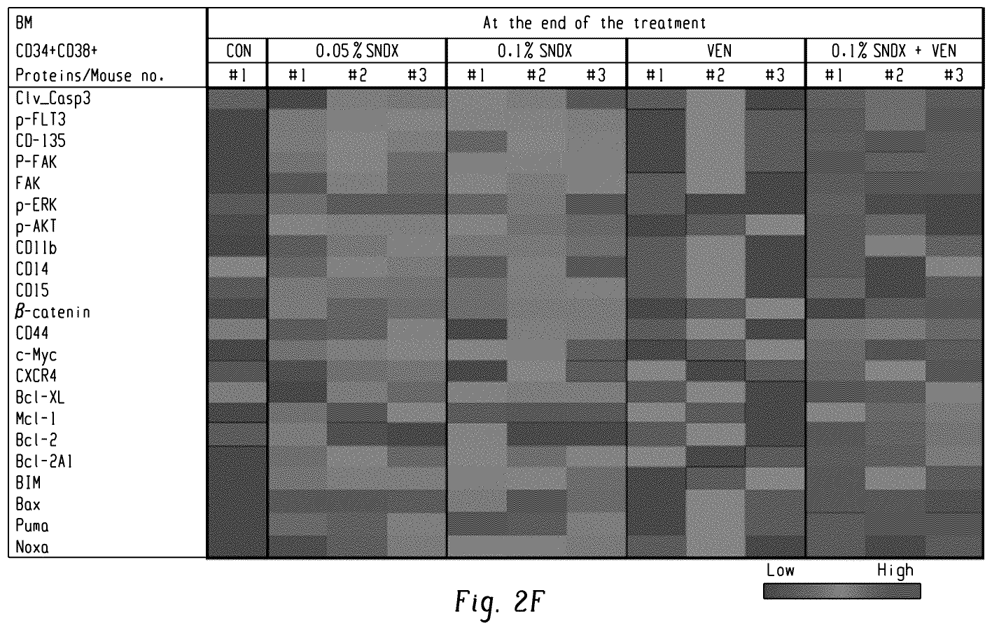

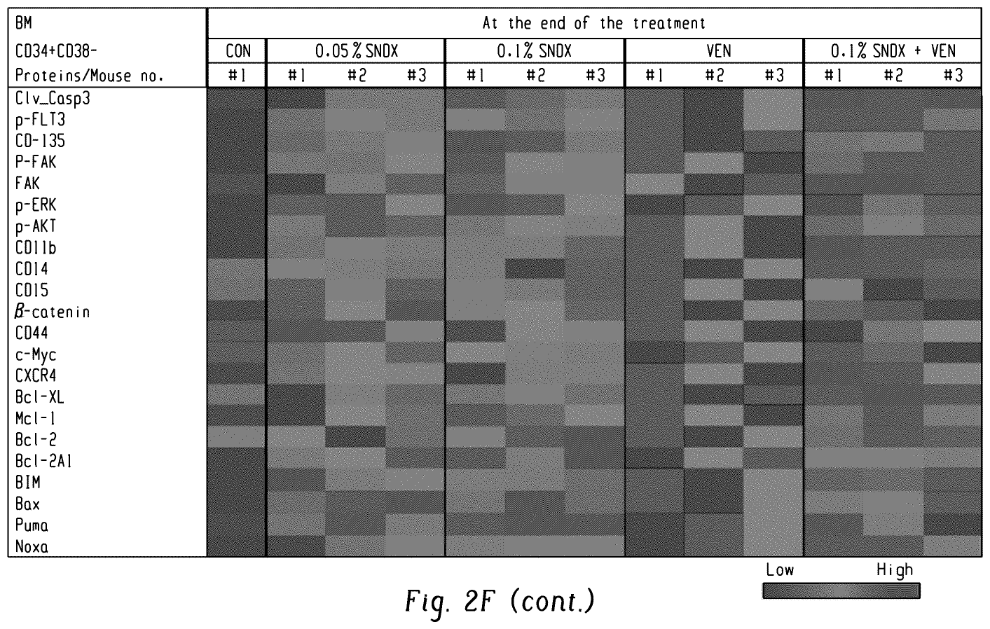

As shown in FIGS. 2A-2E, menin and Bcl-2 inhibition targeted leukemia cells and stem/progenitor cells and modulated Bcl-2 protein levels by CyTOF analysis in BM cells at the end of the treatment. Cell populations were PhenoGraph clustered based on cell surface markers. Cisplatin-low viable single cells were gated with FlowJo (software v10.7, FlowJo LLC) and exported as flow cytometry standard (FCS) data for subsequent analysis in Cytofkit. Cell populations identified and embedded by PhenoGraph in the “Cytofkit_analyzedFCS” files were gated in FlowJo to quantify marker expression. ArcSinh-transformed counts for each protein expression in desired cell populations were visualized with heat maps. Analysis of huCD45⁺ cells shows that Compound I altered the cellular composition and that venetoclax had only minimal effects on leukemia cells, while the combination effectively eliminated the leukemia cells (FIG. 2A). PhenoGraph clustering based on cell surface marker expression grouped huCD45⁺ cells into: CD34⁺CD38⁺, CD34⁺CD38⁺CD123⁺, CD34⁺CD38⁺CD123⁺Tim3⁺, CD34⁺CD38⁻, CD34⁺CD38⁻CD123⁺, and CD34⁺CD38⁻CD123⁺Tim3⁺ populations (FIG. 2B). Compound (I) at 0.05% and more so at 0.1% partially suppressed bulk leukemia cells and effectively targeted CD34⁺CD38⁺/CD34⁺CD38⁺CD123⁺/CD34⁺CD38⁺CD123⁺Tim3 cells. Only at 0.1%, Compound (I) was able to reduce CD34⁺CD38⁻/CD34⁺CD38⁻CD123⁺, but not CD34⁺CD38⁻CD123⁺Tim3⁺ cells. Venetoclax had no activity on bulk leukemia, partial activity in CD34⁺CD38⁺/CD34⁺CD38⁺CD123⁺/CD34⁺CD38⁺CD123⁺Tim3 cells but was active in the CD34⁺CD38⁻/CD34⁺CD38⁻CD123⁺/CD34⁺CD38⁻CD123⁺Tim3⁺ populations. The 0.1% Compound (I) and venetoclax combination was most effective in eliminating all cell types, including leukemia stem/progenitor cells (FIG. 2C). Protein analysis of leukemia cells (FIG. 2D) demonstrated that Compound (I), and more so the combination, decreased Bcl-2 and Bcl-xL, and increased Bim. Furthermore, the combination decreased Bel-2A1, a resistance factor for Bcl-2 inhibition. Protein analysis of CD34⁺CD38⁺ and CD34⁺CD38⁻ cells (FIG. 2F) revealed that Compound (I) increased multiple pro-apoptotic proteins. Compound (I) decreased Bcl-2 in CD34⁺CD38⁺, but not particularly in CD34⁺CD38⁻ cells, which may partially explain its effectiveness in CD34⁺CD38⁺ rather than in CD34⁺CD38⁻ cell populations. The CD34⁺CD38⁺ and CD34⁺CD38⁻ cell numbers were extremely low in the combination-treated group.

Contrary to reports that menin inhibition in NPM1c/FLT3-mutated AML targets FLT3, p-FLT3 was increased in Compound (I)-treated cells, especially in the combination group. Decreased FLT3 expression was observed in vitro in cell lines following short time menin inhibitor treatment, while these results were obtained in vivo in mice treated for one month and reflect the single cell proteomics of surviving cells. The increase in p-FLT3 could be induced by BM environmental factors or could be a resistance mechanism of the surviving cells. Higher levels of pFAK and CD44 may indicate stromal interactions activated to enhance survival. Furthermore, increased huCD11b levels (FIG. 2D) and huCD11b⁺ populations were observed in Compound (I) treated mouse BM cells (FIG. 2E).

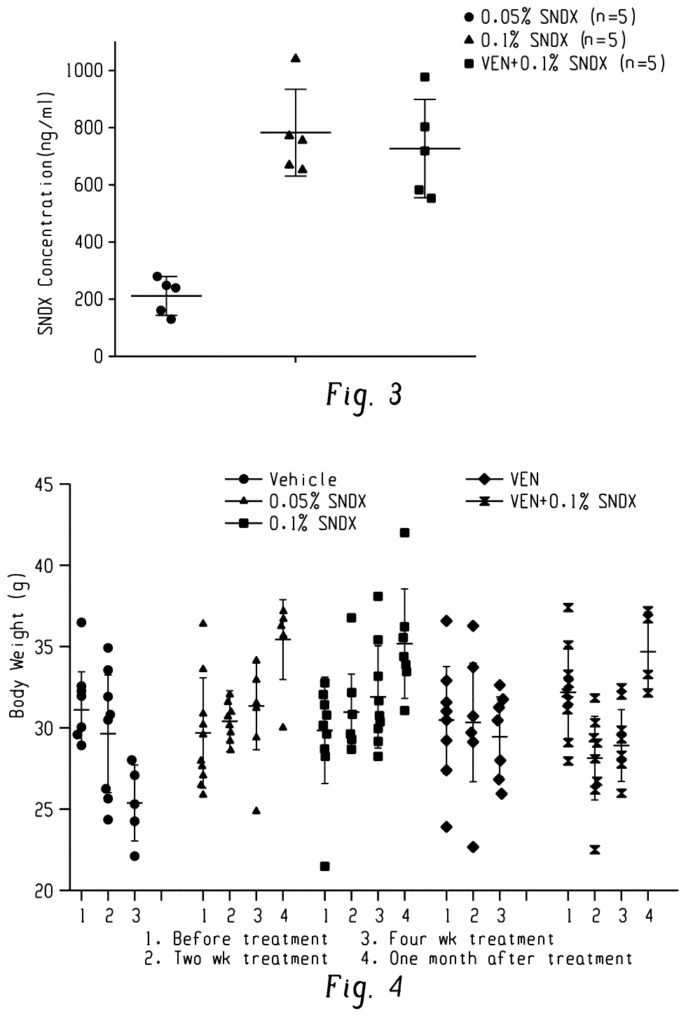

To ensure proper drug intake, blood samples were taken in mice fed with Compound (I)-spiked chow and the drug level was determined in the plasma (n=5). Dose-dependent plasma levels of Compound (I) were observed, which were not affected by treatment with venetoclax (FIG. 3). However, the combination treatment caused weight loss, which could potentially result in an under estimate of combinatorial treatment efficacy. The mice started gaining weight after the treatment was ended (FIG. 4).

Collectively, these data demonstrate that menin inhibition exhibits strong anti-leukemia activity and significantly prolongs mouse survival, which was further enhanced in combination with venetoclax in an NPM1c/FLT3-ITD/TKD AML PDX model. Menin inhibition preferentially targeted CD34⁺CD38⁺ cells, while venetoclax targeted CD34⁺CD38⁻ cells. Only the combined inhibition of menin and Bcl-2 effectively eliminated bulk and CD34⁺CD38⁺/CD34⁺CD38⁻ stem/progenitor cells. Mechanistically, menin inhibition decreased multiple anti-apoptotic Bcl-2 proteins and concomitantly increased pro-apoptotic Bcl-2 proteins that seemingly enhanced the activity of the Bcl-2 inhibitor venetoclax. It is not known if extended treatment would demonstrate further enhanced benefit of this combination. This study further validates menin as a therapeutic target and demonstrates that menin inhibition synergizes with venetoclax in NPM1/FLT3-mutated AML, which warrants further clinical evaluation. Without wishing to be bound by theory, given the high activity of pFLT3 at end of treatment, and the reported synergism of menin and FLT3 inhibition, a triple drug combination may further enhance the activity of menin inhibition in FLT3 mutant AML.

Investigation of the anti-leukemic activity and potential synergism and mechanisms of the combination of the menin-MLL1 inhibitor Compound (I) and venetoclax in vivo in an NPM1c/FLT3-ITD/TKD patient-derived xenograft (PDX) model was performed.

The PDX cell engrafted NSG mice were treated with 0.05 or 0.1% Compound (I)-spiked chow, venetoclax (50 mg/kg), or 0.1% Compound (I) plus venetoclax for one month. Engraftment and disease progression were assessed by flow cytometric measurement of human CD45⁺ cells in mouse peripheral blood. Survival was monitored. The treatment effects on various leukemia cell populations and their protein expression levels were determined by CyTOF mass cytometry.

Menin inhibition exhibited strong anti-leukemia activity and significantly prolonged mouse survival, which was further enhanced when combined with venetoclax, while venetoclax alone had minimal effect. The combination was most effective in extending mouse survival (143 days for 0.1% Compound (I) plus venetoclax, P=0.0003; 131 days and 125 days for 0.1% or 0.5% Compound (I), respectively, P=0.0001 for both; 69 days for venetoclax, P=0.025; vs. 61 days for controls). At the end of treatments, bone marrow cells were collected and CyTOF analysis demonstrated that menin inhibition preferentially targeted CD34⁺CD38⁺ cells, while venetoclax targeted CD34⁺CD38⁻ cells. Only the combined inhibition of menin and Bcl-2 effectively eliminated bulk and CD34⁺CD38⁺/CD34⁺CD38⁻ stem/progenitor cells. Menin inhibition also increased the CD11b⁺ myeloid cell population. Mechanistically, menin inhibition decreased multiple anti-apoptotic Bcl-2 proteins including Bcl-2 and Bcl-xL, and concomitantly increased pro-apoptotic Bcl-2 proteins such as Bax that seemingly enhanced the activity of Bcl-2 inhibition by venetoclax. However, increases of p-FLT3 in the surviving leukemia cells were observed at the end of the treatments, particularly in the combination treated group. Without wishing to be bound by theory, this may contribute to the regrowth of leukemia cells. Synergistic inhibition of Compound (I) with venetoclax in NPM1/FTL3-mediated AML was demonstrated.

### Example 2: Antileukemia Activity of Combined Menin, Bcl-2, and FLT3 Inhibition and a Hypomethylating Agent in NPM1/FLT3-Mutated AML

Example 1 shows that that the menin inhibitor SNDX-50469 (Compound (I)) synergized with the BCL-2 inhibitor venetoclax, but that surviving leukemia cells had increased FLT3 signaling at the end of the treatment. Without being held to theory, it is believed that this increase in p-FLT3 increased MCL-1 and contributed to leukemia outgrowth. Using the same PDX model, we investigated whether FLT3 inhibition with gilteritinib can enhance the efficacy of co-targeting menin and Bcl-2.

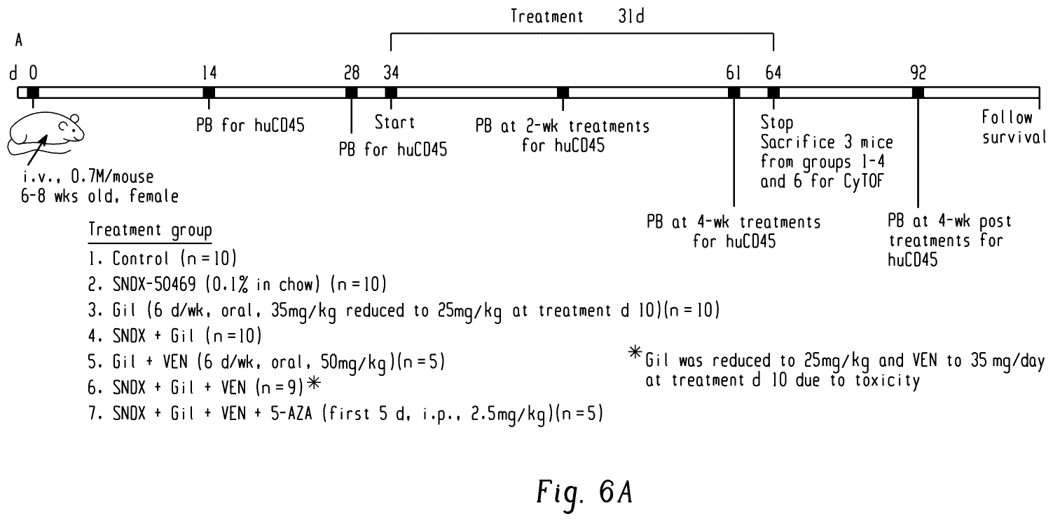

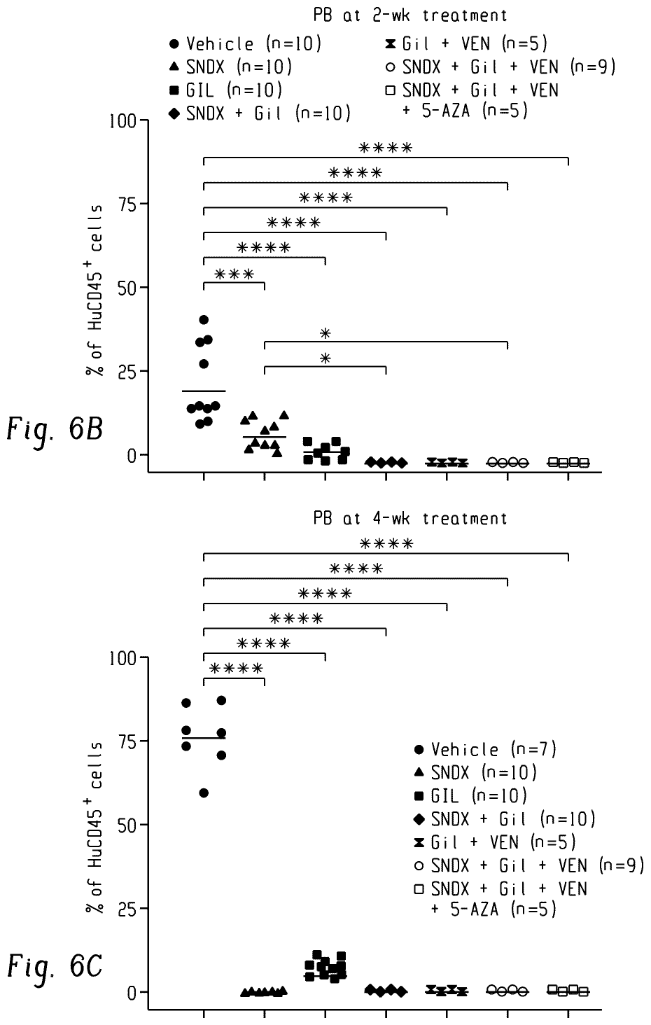

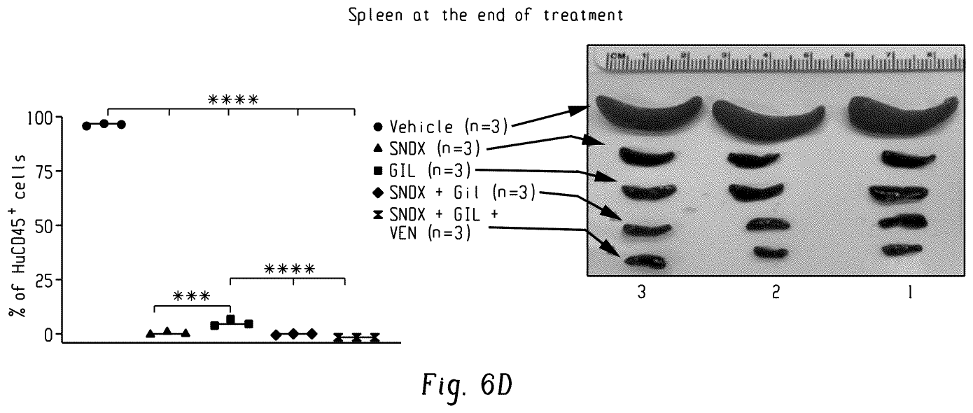

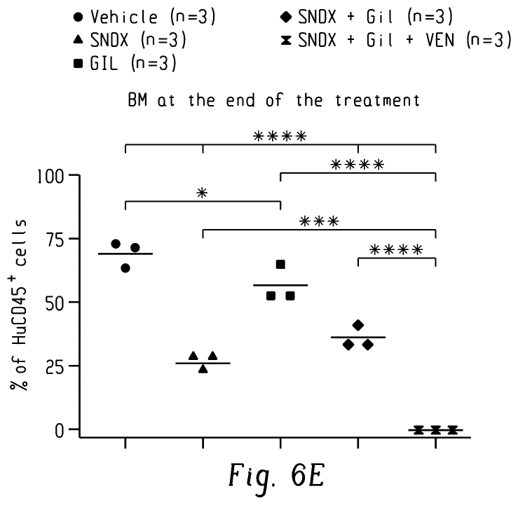

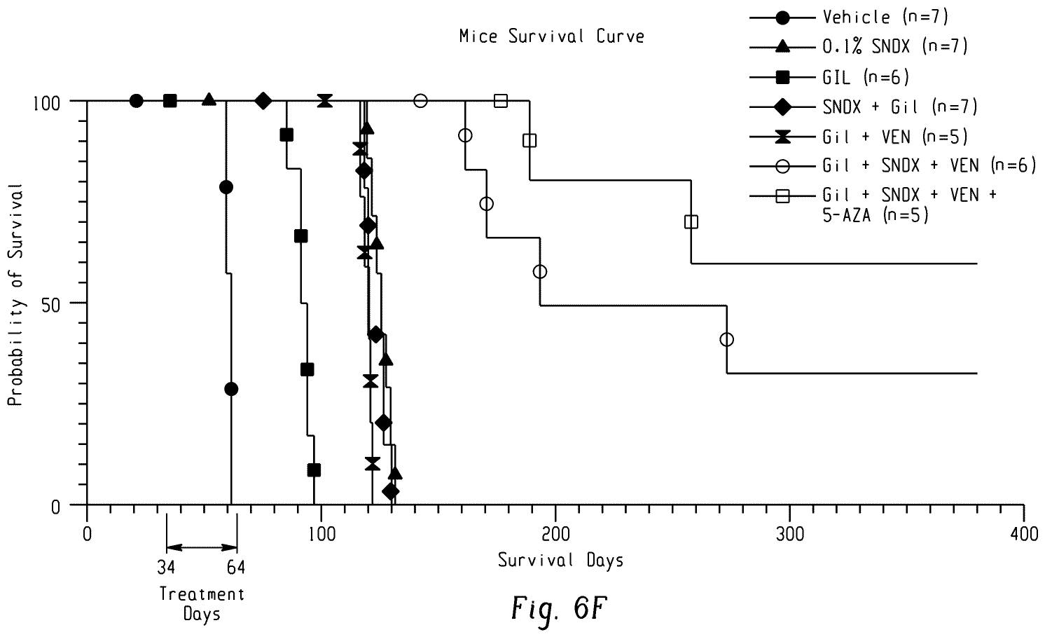

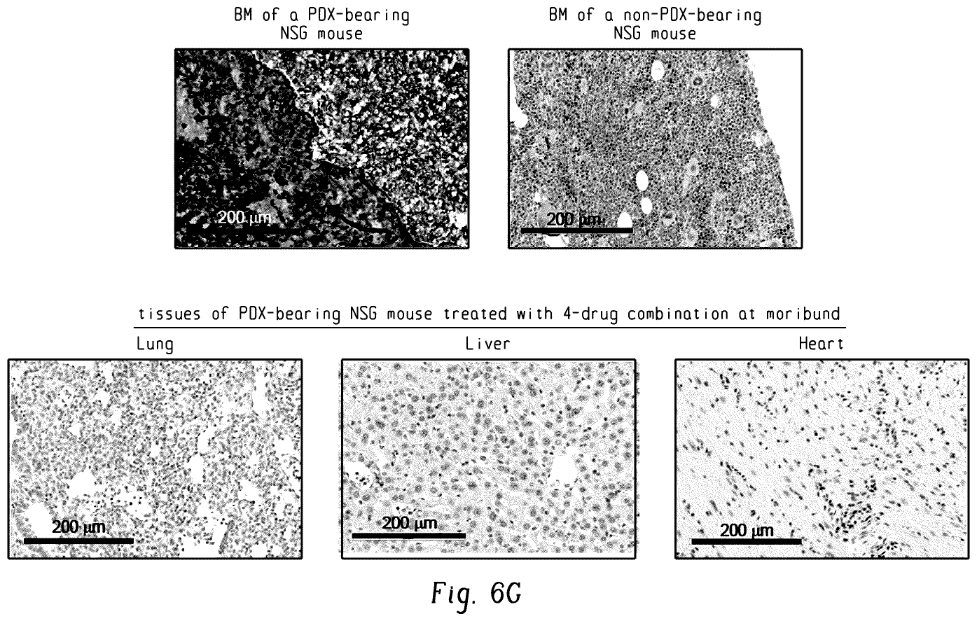

We used the same PDX model (NPM1c/FLT3-ITD/TKD, DFAM-16835) that we used in Example 1. When circulating human CD45 (huCD45) positivity reached 2.6%, the PDX-bearing NSG mice were treated with SNDX-50469 (0.1% in chow), gilteritinib (35 mg/kg), SNDX-50469/gilteritinib, venetoclax (50 mg/kg)/gilteritinib, SNDX-50469/gilteritinib/venetoclax, or SNDX-50469/gilteritinib/venetoclax/5-azacitidine (2.5 mg/kg) (FIG. 6A). Due to rapid weight loss (indicative of toxicity) in mice treated with the 3-drug combination (1 mouse died on treatment day 8) and 4-drug combination (2 mice died on treatment day 7) (excluded in subsequent analysis), we reduced gilteritinib dose from 35 to 25 mg/kg and venetoclax dose from 50 to 35 mg/kg in these two groups beginning on treatment day-10. Reducing the venetoclax and gilteritinib doses prevented further weight loss (data not shown).

Disease progression and treatment response were assessed by flow cytometric measurement and/or immunohistochemical staining of huCD45⁺ cells in peripheral blood or tissues collected at the end of the treatment or at moribund. To assess the effects of treatment on leukemia blasts and phenotypically-defined leukemia stem/progenitor cells and proteins in BM leukemia cell populations, we performed post-treatment CyTOF single-cell proteomics using the antibody panel that we described previously but which also included HOXA9, MEIS1, and PBX3.

At 2 weeks, all treatments significantly lowered circulating huCD45⁺ cells compared to untreated controls, and gilteritinib and gilteritinib/venetoclax greatly enhanced SNDX-50469 activity (FIG. 6B). At 4 weeks, all treatments significantly reduced circulating blasts compared to controls; no significant differences between treatment groups were observed (FIG. 6C). Post-treatment assessments showed that all treatment groups had significantly lower spleen leukemia burden than the control group did; SNDX-50469, SNDX-50469/gilteritinib, and SNDX-50469/gilteritinib/venetoclax were significantly more active than gilteritinib; and SNDX-50469/gilteritinib/venetoclax was more effective than SNDX-50469 and SNDX-50469/gilteritinib but did not reach statistical significance (FIG. 6D). These results were consistent with reductions in spleen size. All treatment groups also had significantly lower BM leukemia burden than controls did; of the treatment groups, gilteritinib was least effective and it did not enhance the activity of SNDX-50469, which was significantly more active than gilteritinib; The percentage of BM leukemia cells in the SNDX-50469/gilteritinib/venetoclax group was significantly lower than those in all other treatment groups (FIG. 6E).

All treatments significantly extended survival (FIG. 6G) compared with controls (median 62 days). SNDX-50469 (128 days) was significantly more effective than gilteritinib (90.5 days; P=0.0001). The control and SNDX-50469-treated mice had survival durations similar to those in our previous study. SNDX-50469/gilteritinib (119 days) did not further improve survival compared to SNDX-50469 alone, possibly because the two agents have overlapping effects on FLT3 signaling. The survival durations of the gilteritinib/venetoclax group (121 days) and SNDX-50469 group did not differ significantly. However, even with the reduced gilteritinib and venetoclax doses, the SNDX-50469/gilteritinib/venetoclax combination extended survival, significantly longer than SNDX-50469, gilteritinib, SNDX-50469/gilteritinib, or venetoclax/gilteritinib did, which was further improved with HMA. The survival duration achieved with the 3-drug or 4 drug combination was much longer than that achieved with SNDX-50469/venetoclax and several mice are still alive over a year in both groups.

Venetoclax alone has limited clinical activity in resistant/relapsed AML, and elderly AML patients have high rates of response to combinations of venetoclax with hypomethylating agents. Therefore, we also treated mice with SNDX-50469/gilteritinib/venetoclax plus 5-azacitidine. The median survival duration of the mice treated with the 4-drug combination (was longer than that of those treated with the 3-drug combination (FIG. 6F, several mice in both groups are still alive one year after)One mouse treated with the 4-drug combination survived 258 days (marked * in FIG. 6F) and had minimal leukemia burden in the BM (0.06%) and spleen (0.15%) and no huCD45⁺ cells in the lungs, liver, or heart (FIG. 6G), suggesting disease cure. Several mice in the 3- and 4-drug combination groups lived close to the life expectancy of normal NSG mice.

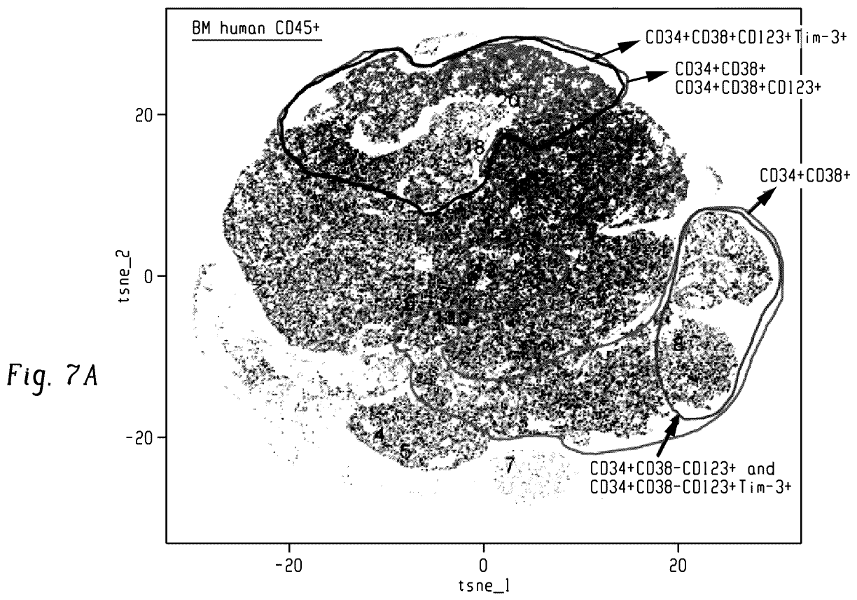

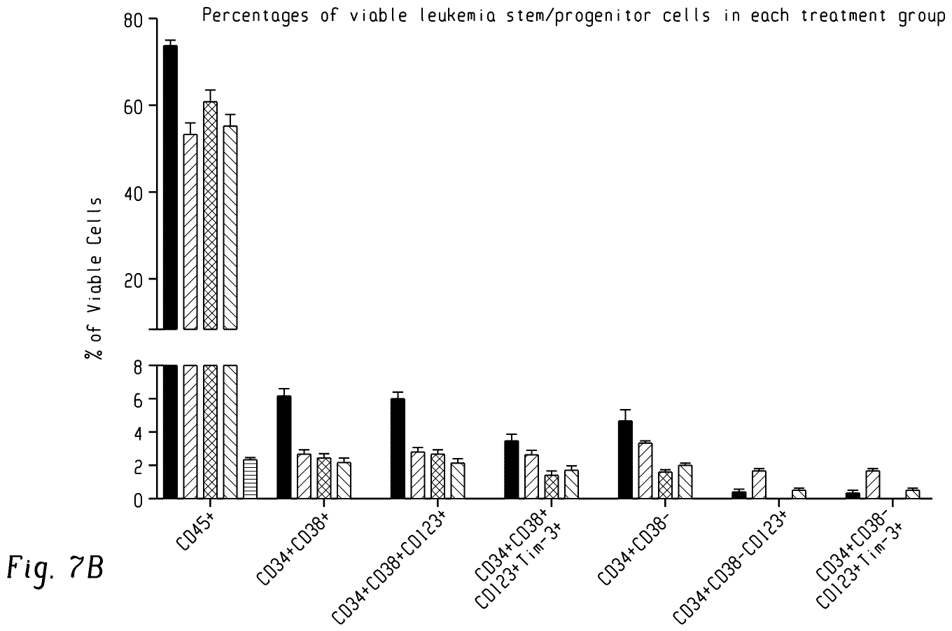

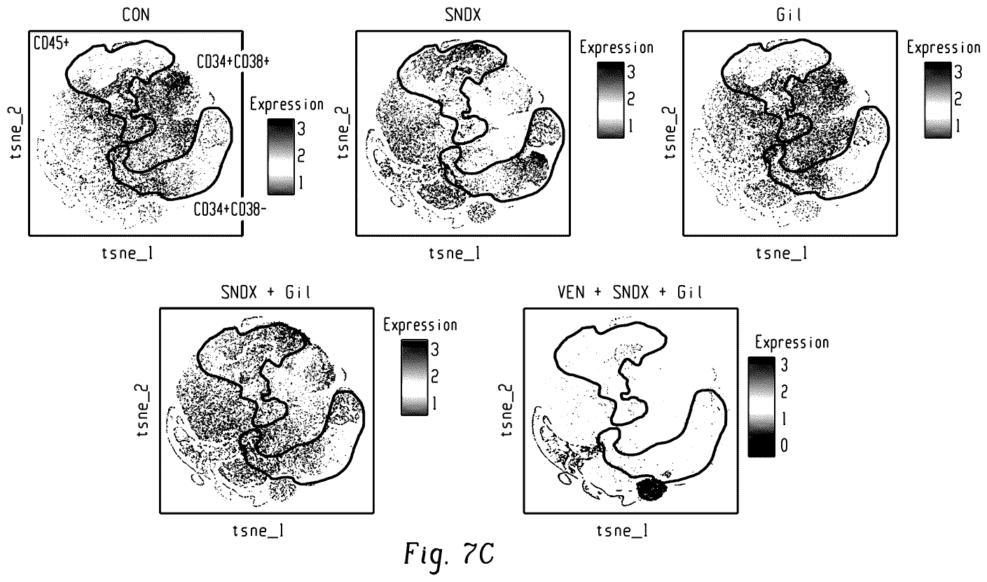

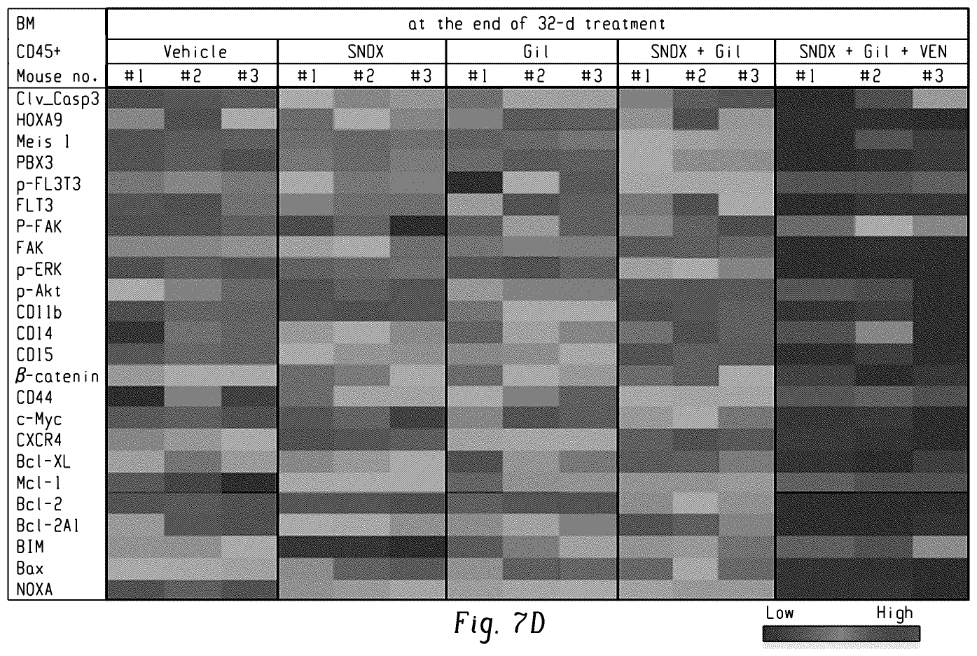

In the CyTOF analysis, leukemia cells were clustered according to cell surface marker expression (FIG. 7A). The percentages of viable leukemia blasts and stem/progenitor cells in the treatment groups and the cell populations in representative mice from each group are shown in FIGS. 7B and 7C, respectively. As we reported previously, SNDX-50469 was more active against CD34⁺CD38⁺ and CD34⁺CD38⁺CD123⁺ populations, except for CD34⁺CD38⁺CD123⁺Tim3⁺ cells, than CD34⁺CD38⁻, CD34⁺CD38⁻CD123⁺, or CD34⁺CD38⁻ CD123⁺Tim3⁺ populations, which were more sensitive to gilteritinib. The SNDX-50469/gilteritinib combination did not exhibit enhanced activity compared with either agent alone, and the 3-drug combination largely diminished leukemia blasts and leukemia stem/progenitor cells.

Protein expression data are shown in FIG. 7D. Consistent with studies showing the effects of SNDX-50469 on RNA levels in Molm13 cells, the CyTOF analysis revealed that both SNDX-50469 and SNDX-50469/gilteritinib greatly decreased MEIS1 and PBX3 protein levels, but had less of an effect on HOXA9, in vivo. SNDX-50469/gilteritinib/venetoclax also decreased HOXA9, and more profoundly than SNDX-50469 alone reduced MEIS1 and PBX3 and suppressed BCL-2, BCL-2A1, and BCL-XL, consistence with the effectiveness of 3-drug combination. As expected, SNDX-50469 increased CD11b.

Our findings demonstrate that the combined inhibition of menin, BCL-2, and FLT3 has strong activity against AML cells and stem progenitor cells and reduces HOX downstream targets and antiapoptotic BCL-2 proteins that conveyed a greater survival benefit, even with the decreased doses than the single or 2-agent treatment in an NPM1c/FLT3-ITD/TKD AML PDX model.

In the 3-drug combination group, residual leukemia cells had increased pFLT3/MCL-1 although FLT3 level was decreased. Whether pFLT3/MCL-1 was inhibited in the 4-drug combination group was not determined. Nevertheless, the addition of 5-azacitidine to the 3-drug combination extended survival significantly. Our data strongly support the clinical evaluation of the combined inhibition of menin, BCL-2, and FLT3 with hypomethylating agents in NPM1/FLT3-mutated AML.

As shown herein, the SNDX-50469/gilteritinib/venetoclax combination had superior activity against leukemia cells and AML stem/progenitor cells and significantly prolonged survival (FIG. 6F; we are still following the survival after over a year), resulting in a survival duration much longer than that achieved with the SNDX-50469/venetoclax combination (FIG. 1G). CyTOF analysis revealed that in addition to BCL-2, SNDX-50469 decreased MEIS1 and PBX3 proteins in vivo and that the 3-drug combination further reduced these proteins. The addition of the hypomethylating agent 5-azacitidine to the 3-part combination further extended survival (FIG. 6F; we are still following the survival after over a year), and this combination potentially eliminated leukemia in some mice. The data support the clinical evaluation of the combined inhibition of menin, BCL-2, and FLT3 with hypomethylating agents in NPM1/FLT3-mutated AML.

What is claimed is:

1. A method of treating cancer with a HOX gene signature in a subject in need thereof, comprising administering to the subject a synergistic combination of a therapeutically effective amount of a menin inhibitor and a therapeutically effective amount of a Bcl-2 inhibitor.

2. The method of claim 1, wherein the menin inhibitor and the Bcl-2 inhibitor are orally administered simultaneously or sequentially.

3. The method of claim 1 or claim 2, wherein the synergistic combination of the therapeutically effective amount of the menin inhibitor and the therapeutically effective amount of a Bcl-2 inhibitor synergistically reduces leukemia CD34⁺CD38⁺/CD34⁺CD38⁻ stem/progenitor cells in bone marrow, synergistically reduces bulk leukemia cells, synergistically decreases anti-apoptotic Bcl-2 protein, improves efficacy compared to a menin inhibitor or a Bcl-2 inhibitor alone, synergistically prolongs survival of the subject, or a combination thereof.

4. The method of claim 1 or claim 2, wherein the synergistic combination of the therapeutically effective amount of the menin inhibitor and the therapeutically effective amount of a Bcl-2 inhibitor synergistically prolongs survival of the subject, wherein the subject has acute myeloid leukemia with one or more AML mutations selected from a nucleophosmin 1 mutation with aberrant cytoplasmic localization (NPM1c), an FLT3 internal tandem duplication (FLT3-ITD), and/or an FLT3 tyrosine kinase domain mutation (TKD).

5. The method of any of the foregoing claims, wherein the therapeutically effective amount of the menin inhibitor, the therapeutically effective amount of the Bcl-2 inhibitor, or both, is reduced compared to the therapeutically effective amount for administration as a single agent.

6. The method of any of the foregoing claims, wherein the menin inhibitor is 5-fluoro-N,N-diisopropyl-2-((4-(7-((trans-4-(methylsulfonamido)cyclohexyl)methyl)-2,7-diazaspiro[3.5]nonan-2-yl)pyrimidin-5-yl)oxy)benzamide, N-ethyl-2-((4-(7-((trans-4-(ethylsulfonamido)cyclohexyl)methyl)-2,7-diazaspiro[3.5]nonan-2-yl)pyrimidin-5-yl)oxy)-5-fluoro-N-isopropylbenzamide, JNJ-75276617, KO-539, DS-1594, DSP-5336, a pharmaceutically acceptable salt thereof, or a combination thereof.

7. The method of claim 6, wherein the menin inhibitor wherein the menin inhibitor is 5-fluoro-N,N-diisopropyl-2-((4-(7-((trans-4-(methylsulfonamido)cyclohexyl)methyl)-2,7-diazaspiro[3.5]nonan-2-yl)pyrimidin-5-yl)oxy)benzamide, or N-ethyl-2-((4-(7-((trans-4-(ethylsulfonamido)cyclohexyl)methyl)-2,7-diazaspiro[3.5]nonan-2-yl)pyrimidin-5-yl)oxy)-5-fluoro-N-isopropylbenzamide and is administered once or twice per day in a daily dose of 200 mg to 600 mg.

8. The method of any of the foregoing claims, wherein the Bcl-2 inhibitor is venetoclax, navitoclax, obatoclax, subatoclax, maritoclax, S64315, oblimersen, or a combination thereof.

9. The method of any of the foregoing claims, wherein the wherein the Bcl-2 inhibitor is venetoclax administered at a daily dose of 20 mg for a first week, at a daily dose of 50 mg for a second week, at a daily dose of 100 mg for a third week, at a daily dose of 200 mg for a fourth week and at a daily dose of 400 mg for a fifth week and subsequent weeks.

10. The method of any of the foregoing claims, further comprising administering a CYP3A inhibitor.

11. The method of any of the foregoing claims, further comprising administering an FLT3 inhibitor.

12. The method of claim 11, wherein the FLT3 inhibitor is midostaurin, sorafenib, sunitinib, lestaurtinib, tandutinib, gilteritinib, quizartinib, crenolanib, or a combination thereof.

13. The method of any of the foregoing claims, further comprising administering a hypomethylating agent.

14. The method of claim 11, wherein the hypomethylating agent is azacitidine, decitabine, guadecitabine, or a combination thereof.

15. The method of any of the foregoing claims, further comprising administering both an FLT3 inhibitor and a hypomethylating agent.

16. The method of any of the foregoing claims, further comprising administering an additional chemotherapeutic agent.

17. The method of claim 16, wherein the additional chemotherapeutic agent comprises cytarabine, 5-fluorouracil, 6-mercaptopurine, capecitabine, floxuridine, fludarabine, gemcitabine, hydroxycarbamide, methotrexate, pemetrexed, phototrexate, or a combination thereof.

18. The method of any of the foregoing claims, wherein the subject has been treated previously with venetoclax for a cancer and the subject progressed on the prior venetoclax treatment.

19. The method of any of the foregoing claims, wherein the subject has been treated previously with venetoclax and developed resistance to venetoclax.

20. The method of any of the foregoing claims, wherein the cancer is a hematological malignancy.

21. The method of claim 20, wherein the hematological malignancy is a lymphoma, a leukemia or multiple myeloma.

22. The method of claim 20, wherein the hematological malignancy is a leukemia.

23. The method of claim 22, wherein the leukemia is acute myeloid leukemia, acute lymphocytic leukemia, myelodysplastic syndrome, chronic myeloid leukemia, or chronic lymphocytic leukemia.

24. The method of claim 23, wherein the leukemia is acute myeloid leukemia.

25. The method of any of claims 22-24, wherein the leukemia is characterized by a mixed lineage leukemia (MLL) rearrangement.

26. The method of any of claims 22-25, wherein the leukemia is characterized by nucleophosmin (NPM1) mutations.

27. The method of any of claims 22-26, wherein the leukemia is further characterized by FLT3 mutations.

28. The method of claim 1, wherein the cancer with a HOX gene signature is breast cancer, multiple myeloma, ovarian cancer, renal cancer, colon cancer, colorectal cancer, prostate cancer, gastric cancer, non-small cell lung cancer, glioblastoma, cervical cancer, chondrosarcoma, osteosarcoma, or neuroblastoma.

29. A therapeutic combination comprising a therapeutically effective amount of a menin inhibitor and a therapeutically effective amount of a Bcl-2 inhibitor.

30. The therapeutic combination of claim 29, further comprising a CYP3A inhibitor, an FLT3 inhibitor, a hypomethylating agent, or a combination thereof.
